(19) United States
(12) Patent Application Publication
(10) Pub. No.: US 2022/0011296 A1
(43) Pub. Date: Jan. 13, 2022

(54) DETERMINING TREATMENT RESPONSE IN SINGLE CELLS

(71) Applicants: Dana-Farber Cancer Institute, Inc., Boston, MA (US); Massachusetts Institute of Technology, Cambridge, MA (US)

(72) Inventors: Keith Ligon, Brookline, MA (US); Seth William Malinowski, Boston, MA (US); Scott R. Manalis, Cambridge, MA (US); Selim Olcum, Cambridge, MA (US); Robert J. Kimmerling, Cambridge, MA (US); Nicholas L. Calistri, New Fairfield, CT (US); David Weinstock, Jamaica Plain, MA (US); Mark Murakami, Cambridge, MA (US); Mark M. Stevens, Seattle, WA (US)

(73) Assignees: Dana-Farber Cancer Institute, Inc., Boston, MA (US); Massachusetts Institute of Technology, Cambridge, MA (US)

(21) Appl. No.: 17/293,890

(22) PCT Filed: Nov. 14, 2019
(86) PCT No.: PCT/US2019/061558

§ 371 Date: May 13, 2021

Related U.S. Application Data
(60) Provisional application No. 62/767,429, filed on Nov. 14, 2018.

(51) Int. Cl. G01N 33/50

(52) U.S. Cl. CPC G01N 33/5017; G01N 33/5085

(57) ABSTRACT

Aspects of the application relate to methods and systems for evaluating treatment response by measuring treatment-induced changes at the single cell level. The disclosure provides methods for isolating single cells that are primary cancer cells, including primary cancer cells from solid tumors, and detecting in minutes to hours from their removal from the body the response of such cells to anti-cancer agents such as radiation, small molecules, biologies, DNA damaging agents and the like.

### RELATED APPLICATION

This application claims the benefit under 35 U.S.C. § 119(e) of U.S. provisional application No. 62/767,429, filed Nov. 14, 2018, the contents of which are incorporated by reference herein in its entirety.

### FEDERALLY SPONSORED RESEARCH

This invention was made with government support, awarded by the National Cancer Institute. The government has certain rights in the invention.

### BACKGROUND

The high level of control offered by microfluidic devices has proven to be valuable for single-cell biological assay development, where measurement of individual cells or small clusters of cells can now be performed with exquisite fidelity. However, for platforms that incorporate on-chip detection, flow rate is governed by the bandwidth required for the measurement, which imposes limitations on the maximum achievable throughput. While throughput can be raised by increasing concentration in some cases, there are often biological and logistical factors that determine the range of achievable sample concentrations. For example, samples processed from primary tissue sources-including biopsies, fine-needle aspirates, blood samples, patient-derived xenograft tissues, and so on-often yield a limited number of cells of interest that set inherent limits on the maximum achievable sample concentration.

Primary cancer cells, as opposed to cancer cell lines, are difficult to grow in culture and tend to change quickly once removed from the body and subjected to culture conditions. They often do not survive very long and die shortly after removal from the body. It would be desirable to be able to test the effects of compounds on primary cancer cells and predict how those compounds might function in the body.

### SUMMARY

Aspects of the application relate to methods and systems for evaluating treatment response by measuring treatment-induced changes at the single cell level. The disclosure provides methods for isolating single cells that are primary cancer cells, including primary cancer cells from solid tumors, and detecting in minutes to hours from their removal from the body the response of such cells to anti-cancer agents such as radiation, small molecules, biologics, DNA damaging agents and the like. The disclosure further provides for multiple detections, different from one another, on the same single cell, which may be carried out substantially simultaneously or serially and which detections may be combined in characterizing the sensitivity of the cell to anti-cancer agents or for otherwise characterizing the primary cancer cell. The disclosure provides for detections including detecting the effect of the anti-cancer agent on the mass of a primary cancer cell from a subject, such detection being measured over very short periods of time and used to predict the in vivo effect of such anti-cancer agent on the primary cancer cells in the subject. Mass can be combined with other markers such as mass rate of change, cell surface markers, and other characteristics of the cell. The ability to make such predictions based on tests of live, primary cancer cells obtained from a solid tumor of a subject was heretofore unknown.

For example, the disclosure provides a method of predicting sensitivity of a cancer cell to a cytotoxic agent by obtaining primary cancer cells from a subject, which may be from a solid tumor, separating the cancer cells from one another and causing at least some of the cancer cells to pass individually and separately in time through a channel in a microfluidics device, the channel adapted to measure the mass of a cell as it passes through the channel, contacting one of the primary cancer cells with a cytotoxic agent, detecting the mass of the cell contacted with the cytotoxic agent as it passes through the channel after it has been contacted with the cytotoxic agent, in one embodiment detecting mass numerous times over a period of time, and comparing the mass of the cell to the mass of a control cell, which control cell may be one of the primary cancer cells that has not been contacted with the cytotoxic agent. A decrease in the mass of the cell contacted with the cytotoxic agent versus the cell not contacted with the cytotoxic agent indicates that the primary cancer cells are sensitive to the cytotoxic agent.

In some aspects a method for evaluating sensitivity of a cancer cell to an anti-cancer reagent is disclosed. The method involves (a) obtaining a tissue sample comprising primary cancer cells from a subject; (b) dissociating the tissue sample into single primary cancer cells; (c) contacting the single primary cancer cells with an anti-cancer reagent; and (d) detecting the mass of the single primary cancer cell contacted with the anti-cancer reagent as it passes through a channel, wherein the mass of the cell contacted with the anti-cancer reagent is compared to the normalized mass of a control cell that is not contacted with an anti-cancer reagent.

In other aspects a method for identifying an anti-cancer reagent is provided. The method involves (a) obtaining a tissue sample comprising primary cancer cells from a subject; (b) dissociating the tissue sample into single primary cancer cells; (c) contacting the single primary cancer cells with a reagent; and (d) detecting the mass of the single primary cancer cell contacted with the reagent as it passes through a channel, wherein if the normalized mass of the cell contacted with the reagent is less than a control cell that is not contacted with the reagent, the reagent is an anti-cancer reagent.

In other aspects a method for evaluating sensitivity of a cancer cell to an anti-cancer reagent is provided. The method involves (a) obtaining a tissue sample comprising primary cancer cells from a subject; (b) dissociating the tissue sample into single primary cancer cells; (c) culturing the single primary cancer cells to obtain patient-derived cell lines; (d) contacting the patient-derived cell lines with an anti-cancer reagent; (e) engrafting a host subject with the patient-derived cell lines contacted with the anti-cancer reagent; (f) obtaining a tissue sample from the host subject; (g) dissociating the tissue sample from the host subject into single cells; and (h) detecting the mass of the single cells contacted with the anti-cancer reagent as they passes through a channel, wherein the mass of the cell contacted with the anti-cancer reagent is compared to the normalized mass of a control cell that is not contacted with an anti-cancer reagent.

In some embodiments when the mass of the cell contacted with the anti-cancer reagent is decreased compared to the control cell, the cancer cell is sensitive to the anti-cancer reagent. In other embodiments when the mass of the cell contacted with the anti-cancer reagent is the same or increased compared to the control cell, the cancer cell is resistant to the anti-cancer reagent.

In some embodiments steps (b)-(d) are performed within one hour to one month after step (a). In other embodiments the single primary cancer cells of step (b) are cultured to produce patient-derived cell lines. In some embodiments the patient-derived cell lines are subjected to steps (c) and (d).

In some embodiments the patient-derived cells lines are engrafted into a host subject, thereby generating a patient-derived xenograft. In some embodiments dissociating the tissue sample comprises enzymatic and/or physical dissociation. In some embodiments the anti-cancer reagent comprises radiation, small molecules, biologics, and/or DNA damaging agents.

In some embodiments the channel for detecting the mass of the single primary cancer cell is a measurement channel. In some embodiments the single cells are flowed into and through the measurement channel by active loading.

In some embodiments the single cells are classified as single cells, cell aggregates, or debris in real-time before they are flowed into the measurement channel. In some embodiments the classification is at least 85% accurate at allowing only single cells into the measurement channel compared to manual classification. In other embodiments the classification is at least 50% accurate at rejecting cell aggregates and debris from the measurement channel compared to manual classification.

In some embodiments the contacting in step (c) is for 1-10 days.

The details of certain embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1A shows a sample processing pipeline for serial suspended microchannel resonator (sSMR) measurement with active loading in an example process of ex vivo drug sensitivity testing of patient resections. Tumor cells were isolated from patient resection specimens using established protocols (see, e.g., EXAMPLES of the application) for dissociation into single-cell suspension and allowed to recover for at least 24 hours before the addition of drug or vehicle control. On subsequent days, the buoyant mass and mass accumulation rate (MAR) were measured for both the control and drug-treated fractions.

FIG. 1B is a Tukey's box plot showing the buoyant mass measurements for primary biopsies of different brain lesions. From left to right, number of cells measured: n=86, 90, 63, 64, 66, 83, 74, 60, 47, 53, 54, 164, and 188. The center line shows median value, hinges represent the first and third quartiles, and whiskers extend to the furthest value <1.5×IQR from hinge.

FIG. 1C is a Tukey's box plot showing mass-normalized MAR values from the same primary tissue samples shown in FIG. 1B. Statistically significant reductions in MAR per mass were observed for the recurrent glioblastoma treated with 1 μM abemaciclib for 72 hours (p=0.032), breast metastasis treated with 100 nM abemaciclib (p=0.029), and lung metastasis treated with 100 μM carboplatin (p=0.025). All other drug-control comparisons did not show a statistically significant response. The center line shows median value, hinges represent the first and third quartiles, and whiskers extend to the furthest value <1.5×IQR from hinge.

FIG. 1D shows results for rare cell measurement of BaF3 cells. Plot (I) is a dot plot of raw mass versus time data for BaF3 cells measured at each cantilever in a 12 cantilever sSMR device. Shaded dots represent each individual cantilever, with the progression proceeding from black to dark gray to light gray to medium gray moving from the first to the last cantilever on the flow path. Single-cell trajectories are subjected to a linear fit to extract MAR. Cells were seeded by serial dilution at a density of 2.7×10³ cells/mL, with ˜270 total cells in 100 μL. 165 of the 270 cells (61%) were loaded into the array after 3 hours of measurement. Plot (II) is a dot plot of MAR versus mass for the same BaF3 cells.

FIG. 2A shows a schematic of active loading by optically triggered fluidic state switching. Regions of interest (ROIs) are labeled as boxes. ROI 1 (top-most box) is used to detect particles when in the “seek” state. Detection of a particle traveling at a high flow rate in the sampling channel by ROI 1 causes a temporary change to the default “load” state, and reverts following entrance of a single particle into the measurement channel as detected by ROI 4 (box within ROI 3). ROI 2 (box within ROI 1) maintains the presence of a single particle in the sampling channel for the next loading duty cycle. As a single particle is detected by ROI 2 while in the “load” state, it triggers adoption of a “queue” state, which bumps the cell back in the sampling channel before reverting to the “load” state. This continues until the duty cycle is complete. ROI 4 and ROI 3 (bottom-most box) work together to detect entrance into the measurement channel and the presence of debris or doublet events, respectively. Once ROI 4 detects entrance of a particle in the “load” state, ROI 3 quickly images the event, switching to the “reject” state if the particles geometry or contrast is outside previously set parameters defining an unwanted particle.

FIG. 2B shows a comparison between passive throughput (22 cells h⁻¹, 95% CI: 13, 39, n=9) and active loading (386 cells h⁻¹, 95% CI: 354, 433, n=247) for murine L1210 cells (50 μL⁻¹) flowing through a transit time detector in the measurement channel. Zoom-in plots show passage of a single cell with a predefined transit time of ˜800 ms.

FIG. 2C shows a schematic of a sSMR platform. The device consists of an array of SMR buoyant mass sensors placed periodically along the length of a long (50 cm) microfluidic measurement channel. The array is flanked on either side with two sampling channels with independent control of upstream and downstream pressures. For single-cell transit time measurements, the first cantilever of the sSMR was used to detect cell entrance in to the array (inset). The schematic of this cantilever demonstrates a cell flowing through the cantilever (left) and the corresponding resonant frequency measurements associated with these positions (right).

FIG. 2D is a representative plot showing the single-cell frequency measurements at various stages of filtering. The binary occupancy readout (solid line, top plot), shown here with the same time scale as the frequency data, indicates when the frequency shift is below the specified occupancy threshold (dashed line).

FIG. 3A shows a schematic of the sSMR. Sampling channels on either side of the device (100 m wide and 30 m deep) are each accessed via two ports with independent pressure control to achieve the fluidic states presented in FIG. 3B. These sample channels are connected with a serpentine channel (50 cm long, 20 m wide, and 25 m high) with 10-12 SMR mass sensors spaced evenly along its length. MAR is calculated by taking the slope of the linear least squares fit of mass measurements collected from individual SMRs as a function of time for each single-cell trajectory.

FIG. 3B depicts COMSOL models demonstrating the flow characteristics of the four different fluidic states presented in FIG. 2A and described in Example 3 of the application. The model shows the T-junction entrance of the sSMR, outlined with a red box in FIG. 3A. Flow patterns were modeled using the volumetric flow rates described in Example 1 of the application to recapitulate experimental conditions.

FIG. 3C shows a comparison of theoretical throughput limits (solid and dashed lines for active and passive loading, respectively) with experimental results (solid points and open squares for active and passive loading, respectively) for samples with 1, 10, 50, 100, and 1000 L1210 cells μL⁻¹ (n=15, 105, 143, 149, and 83 for active loading and n=1, 8, 64, 87, and 309 for passive loading) collected with a 15 second minimum spacing. The theoretical model is based on a 15 second duty cycle (e.g., as described in Example 1 of the application). Measurement error bars represent the 95% CI (two-tailed t test) of loading period (s) converted to throughput (events h⁻¹). Each concentration was measured continuously for at least 20 min. The passive loading sample at 1000 cells μL⁻¹ had a throughput of 747 cells h⁻¹, 95% CI: 673, 832.

FIG. 3D is a dot plot of MAR versus mass comparing L1210 cells measured from standard, growth-phase culture concentrations (100 cells μL⁻¹, closed circles, n=426), or from samples with low concentration and low total cell count (˜2 cells μL⁻¹, 100 total cells, open circles, n=47).

FIGS. 4A-4D illustrate various aspects relating to particle classification in a microfluidics system. FIG. 4A shows an example of automated particle classification. Panels (I) through (IV) depict examples of automatically classified particles, and panel (V) is a particle classification diagram depicting the automated particle classification logic. FIG. 4B illustrates change of flow rate in the sampling channel as a function of time during a cell loading cycle. FIG. 4C shows a plot of the throughput improvement for a range of sample concentrations in different systems, and the specifications used for calculating improvement in each system. FIG. 4D shows throughput improvement (numbers in bold) for applying active loading to previously published single-cell measurements. Throughput improvement is defined by the ratio between the effective sampling flow rate and the flow rate that would have been achieved in the measurement channel without active loading. A value of unity indicates that there would be no improvement from active loading. FIG. 4E shows a plot illustrating throughput modeling with desired minimum particle spacing. FIG. 4F shows plots of accuracy of real-time cell classification used for active loading. FIG. 4G is a flow chart that depicts a fluidics process in accordance with the application.

FIGS. 5A-5L show results from cell mass and MAR measurements obtained for a diverse range of clinical brain tissue and cancer samples exposed to either a standard-of-care therapy or experimental therapy currently in clinical trial. FIG. 5A shows results obtained using non-tumor brain tissue resected for a non-tumor condition, and FIG. 5B shows representative images of accepted and rejected non-tumor cells. FIG. 5C shows results obtained using primary glioblastoma, and FIG. 5D shows representative images of accepted and rejected primary glioblastoma cells. FIG. 5E shows results obtained using recurrent glioblastoma, and FIG. 5F shows representative images of accepted and rejected recurrent glioblastoma cells. FIG. 5G shows results obtained using metastatic breast adenocarcinoma, and FIG. 5H shows representative images of accepted and rejected breast adenocarcinoma cells. FIG. 5I shows results obtained using metastatic non-small-cell lung cancer, and FIG. 5J shows representative images of accepted and rejected metastatic non-small-cell lung cancer cells. FIG. 5K shows results obtained using primary central nervous system (CNS) lymphoma, and FIG. 5L shows representative images of accepted and rejected primary CNS lymphoma cells.

FIGS. 6A-6F show an overview of the data acquisition pipeline to obtain single cell mass accumulate rate (MAR) data in response to chemotherapy treatment. FIG. 6A shows a patient tumor resection, in which the tissue is brought to the research lab for dissociation. FIG. 6B shows parallel tissue diagnosis and pathology reports. FIG. 6C depicts the process for acute patient sample testing using a single cell mass as readout. The tumor tissue is dissociated into single cells, and mass/MAR of the single cells in response to chemotherapeutic agents can be measured within a week of resection. FIG. 6D shows the measurement and analysis of long term, patient derived cell lines (PDCLs). FIG. 6E shows PDCLs being implanted in vivo to allow mass measurements to be taken ex vivo from treated mice. FIG. 6F shows an example full complete dataset, including the novel single cell mass readouts.

FIGS. 7A-7D show single cell MAR results generated using the SMR pipeline in both PDCLs and acute patient models. FIG. 7A shows MAR data generated from a heterogeneous cohort of PDCLs. The x-axis is time after chemotherapy treatment and the y-axis is the MAR of single cells. FIG. 7B shows that single cell MAR is effective biomarker for determination of treatment based on ex vivo chemosensitivity in cancer types. FIG. 7C shows single cell MAR data from acutely dissociated and TMZ treated patient tissue samples from surgery. FIG. 7D shows that single cell MAR measurements detect resistance to chemotherapy.

### DETAILED DESCRIPTION

Described herein are devices, methods, and systems for assessing cell properties, such as mass in response to stimuli such as putative therapeutic agents. A microfluidics-based system is used to quantify cellular properties that provide information about the responsiveness of a cell to a reagent that yields important information about a cell or tissues ability to respond to a particular treatment. Exemplary uses of the devices provided herein are included in the description, claims and Examples below. However, these uses are not meant to be limiting and additional uses would be apparent to the skilled artisan based on this disclosure. The Examples provided herein relate to cancer cells in order to demonstrate the effectiveness of the devices, systems and methods described herein on that cell population. However, the invention is not limited to cancer cells. Other pathologies may be examined using the devices, methods and systems provided herein. Briefly, the Examples demonstrate that the devices, systems and methods can be used to, in a high throughput multiplex format, identify the mass of individual cells that have been exposed to a potentially therapeutic reagent and compare that mass with the mass of a control cell in order to determine the impact the reagent had on the treated cell. These results point to the use of these devices, systems, and methods for a number of applications, including but not limited to, screening for and identifying therapeutics, assessing a patient response to a therapeutic, determining the effectiveness of a therapeutic, and diagnosing a subject with a disease or condition, as well as research applications.

Microfluidic devices are provided herein for evaluating, characterizing, and/or assessing properties of cells, such as cell mass under controlled single cellular pressure based conditions. In particular, devices are provided for measuring, evaluating and characterizing dynamic mechanical responses of biological cells, e.g., cancer cells, to therapeutic agents. The devices are typically designed and configured to permit measurements of cell mass in a high throughput manner. For example, by measuring the mass of cells treated with different agents, the responsiveness of the cells to various reagents can be assessed in a rapid high throughput manner that could not previously be achieved.

Thus, in some aspects, the present disclosure provides methods for evaluating sensitivity of a cancer cell to an anti-cancer reagent. A cancer cell is sensitive to an anti-cancer reagent when the cancer cell is killed or the growth and/or spread of the cancer cell is inhibited by contacting the cancer cell with the anti-cancer reagent. Cancer cells may also be resistant to an anti-cancer reagent, wherein contacting the cancer cell with the anti-cancer reagent does not kill or inhibit the growth and/or spread of the cancer cell. Resistance may be inherent to the cancer cell, wherein the anti-cancer reagent never kills or inhibits the growth and/or spread of the cancer cell. Resistance may also be acquired, wherein the cancer cell is initially sensitive to the anti-cancer reagent, but over time the cancer cell becomes resistant. Sensitivity of a cancer cell to an anti-cancer reagent may be determined based on any method known in the art including, cell mass, proliferation, survival, metastasis, and/or expression of cell surface markers.

In some embodiments, when the cancer cell is sensitive to the anti-cancer reagent, the mass of the cell contacted with the anti-cancer reagent decreases compared to a control cell. A control cell is a normal (e.g., non-cancerous cell). A control cell may be another cell derived from the same tissue in the subject that does not comprise cancer cells, a cell that was previously cancerous but is no longer cancerous, or a cell from another subject that is derived from the same tissue type as the cancer cell. In some embodiments, when the cancer cell is resistant to the anti-cancer reagent, the mass of the cell contacted with the anti-cancer reagent increases or stays the same compared to the control cell. In order to compare the mass of single cells (e.g., primary cancer cells versus control cells), the mass of the single cells being compared is normalized.

In some embodiments, the methods comprise obtaining a tissue sample comprising primary cancer cells from a subject. Obtaining a tissue sample may be by any method known in the art including, but not limited to, solid tumor biopsy, non-solid (e.g., blood) liquid biopsy, bone biopsy, hollow-needle biopsy, and aspiration. Tissue samples may be from cancerous tissues (e.g., comprising cancer), non-cancerous tissues (e.g., normal tissues) or mixed cancerous tissues and non-cancerous tissues. Tissue samples are obtained from non-cancerous tissues that are the same type of tissue as cancerous tissues (e.g., cancerous brain tissue and normal brain tissue). Tissue samples may be obtained from any tissue including, but not limited to, brain, blood, lung, breast, colon, stomach, nervous, pancreas, liver, bone marrow, spleen, bone, small intestine, rectum, esophagus, trachea, and skin.

In some embodiments, the tissue sample comprises primary cancer cells. Primary cancer cells are cancer cells that are obtained from a subject having cancer. A subject may be any mammal that has cancer. Non-limiting examples of subjects include humans, mice, rats, non-human primates, dogs, cats, pigs, and cattle. In some embodiments, the cancer is a primary cancer, in which the subject has not previously had the cancer and/or the cancer has not been treated. In some embodiments, the cancer is a relapsed cancer, in which the cancer has recurred in a subject that previously had cancer that was treated and went into remission. In some embodiments, the recurrent cancer is the same type (e.g., brain, lung, etc). as the primary cancer.

Some aspects of the present disclosure provide methods of detecting the mass of a single cell (e.g., primary cancer cell) as it passes through a channel. Detecting the mass may be by any method known in the art including, but not limited to, microcantilever-based microbiosensors, optical quantitative phase imaging, pedestal resonant sensors, and suspended microchannel resonators. In some embodiments, the detecting is performed using a microcantilever-based microbiosensor as described in the Examples.

The microcantiliver-based microbiosensor (MBM) may comprise multiple channels, a pump for moving fluid comprising the single cells through the channel, and a detector. In some embodiments, the MBM comprises a sample channel in which the particles in samples comprising the single cells (e.g., cancer cells, normal cells) are classified into different categories based on size. The different categories include, but are not limited to: single cells (e.g., singlets), cell aggregates (e.g., doublets and multiple singlets), and debris. This categorization ensures that only single cells are examined to detect their mass.

In some embodiments, once single cells (e.g., cancer cells, normal cells) are categorized in the sample channel, the fluid sample containing the single cells is flowed through a measurement channel to detect the mass of the single cells. Detection of the mass may be by any method known in the art including, but not limited to: resonant frequency, duration of time in the measurement channel, and diffraction of light. In some embodiments, the mass of single cells is determined by resonant frequency. This technology is summarized in Bryan, et al., 2014, Measuring single cell mass, volume, and density with dual suspended microchannel resonators, Lab Chip, 14(3): 569-576, the contents of which is incorporated herein in its entirety. Briefly, the microfluidic device consist of at least one fluid channel embedded in a vacuum-packaged cantilever. The cantilever resonates at a frequency proportional to its total mass, and as a single cell travels through the channel, the total cantilever mass changes. This change in mass is detected as a change in resonance frequency that corresponds directly to the buoyant mass of the cell. If the same cell is measured a second time in a fluid with a different density, then a second buoyant mass is obtained. From these two measurements the mass, volume, and density of a single cell may be calculated.

In some embodiments the device comprises a suspended microchannel resonator (SMR).

SMRs are resonant mass sensors that contain liquid within the mechanical structure, thereby minimizing damping associated with the fluidic viscous drag. The SMR may be serial suspended microchannel resonators (sSMR) in some embodiments. The disclosure further provides for multiple detections, different from one another, on the same single cell, which may be carried out substantially simultaneously or serially and which detections may be combined in characterizing the sensitivity of the cell to anti-cancer agents or for otherwise characterizing the primary cancer cell. These multiple detection steps can be performed in a high throughput manner in a sSMR device.

In some cases, the methods described herein are designed such that a single cell may be isolated from a plurality of cells and flowed into a fluidic channel (e.g., a microfluidic channel). For example, the single cell may be present in a plurality of cells of relatively high density and the single cell is flowed into a fluidic channel, such that it is separated from the plurality of cells. In some cases, more than one cell may be flowed into a fluidic channel such that each cell enters the fluidic channel at a relatively low frequency (e.g., of less than 1 cell per 10 seconds). The cells may be spaced within a fluidic channel so that individual cells may be measured/observed over time.

Any of the microfluidic channels of the present disclosure may have a size to accommodate a cell or cells. For instance the channels may have a height, for example from a top wall to a bottom wall, ranging from 0.5 μm to 100 μm. The microfluidic channel of any of the devices provided herein may have a height in a range of 0.5 μm to 100 μm, 0.1 μm to 100 μm, 1 μm to 50 μm, 1 μm to 50 μm, 10 μm to 40 μm, 5 μm to 15 μm, 0.1 μm to 5 μm, or 2 μm to 5 μm. The microfluidic channel may have a height of up to 0.5 μm, 1 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10 μm, 20 μm, 30 μm, 40 μm, 50 μm, 75 μm, 100 μm, or more. In a specific embodiment, the microfluidic channel has a height of 15 μm, or about 15 μm.

Any of the microfluidic channels of the present disclosure may have a width, for example from a first side wall to a second side wall, ranging from 0.01 mm to 5 mm. The microfluidic channel of any of the devices provided herein may have a width in a range of 0.01 mm to 4 mm, 0.1 mm to 3 mm, 0.1 mm to 2 mm, 0.2 mm to 2 mm, 0.5 mm to 2 mm, 0.5 mm to 1.5 mm, 0.8 mm to 1.5 mm, or 1 mm to 1.4 mm. In some embodiments, the microfluidic channel may have a width of up to 0.01 mm, 0.05 mm, 0.2 mm, 0.4 mm, 0.6 mm, 0.8 mm, 1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.8 mm, 3 mm, 3.5 mm, 4 mm, 4.5 mm, 6 mm, 6.5 mm, 7 mm, or more. In a specific embodiment, the microfluidic channel has a width of 1.3 mm, or about 1.3 mm.

Devices containing a microfluidic channel can further contain a substantially planar transparent wall that defines a wall of a microfluidic channel. This substantially planar transparent wall, which can be, for example, glass or plastic, permits observation into the microfluidic channel by microscopy so that at least one measurement of each cell that passes through one of the microfluidic channels can be obtained. In one example, the transparent wall has a thickness of 0.05 mm to 2 mm. In some cases, the transparent wall may be a microscope cover slip, or similar component. Microscope coverslips are widely available in several standard thicknesses that are identified by numbers, as follows: No. 0-0.085 to 0.13 mm thick, No. 1-0.13 to 0.16 mm thick, No. 1.5-0.16 to 0.19 mm thick, No. 2-0.19 to 0.23 mm thick, No. 3-0.25 to 0.35 mm thick, No. 4-0.43 to 0.64 mm thick, any one of which may be used as a transparent wall, depending on the device, microscope, cell size, and cell detection strategy.

The device described above can further contain a reservoir fluidically connected with the one or more microfluidic channels, and a pump that perfuses fluid from the reservoir through the one or more microfluidic channels, and optionally, a microscope arranged to permit observation within the one or more microfluidic channels. The reservoir may contain cells suspended in a fluid. The fluidics connecting the reservoir to the microfluidic channel may include one or more filters to prevent the passage of unwanted or undesirable components into the microfluidic channels.

In some cases, the methods may be carried out in a high throughput manner. In some aspects, methods are provided that are useful for diagnosing, assessing, characterizing, evaluating, and/or predicting disease based on transit characteristics of cells, e.g., cancer cells, and tissues, in microfluidic devices. In one aspect, the present disclosure includes a high throughput method of measuring a morphological and/or mechanical property of an individual cell such as mass.

In exemplary embodiments the methods are performed on cancer cells to determine the impact of a cytotoxic agent on the cancer cell. The methods may be performed on a microfluidics device such as an sSMR by separating cancer cells isolated from a patient from one another and causing at least some of the cancer cells to pass individually and separately in time through a channel in the device, the channel adapted to measure the mass of a cell as it passes through the channel, contacting one of the primary cancer cells with a cytotoxic agent, detecting the mass of the cell contacted with the cytotoxic agent as it passes through the channel after it has been contacted with the cytotoxic agent, in one embodiment detecting mass numerous times over a period of time, and comparing the mass of the cell to the mass of a control cell, which control cell may be one of the primary cancer cells that has not been contacted with the cytotoxic agent.

The disclosure provides for detections including detecting the effect of the anti-cancer agents on the mass of a primary cancer cell from a subject, such detection being measured over very short periods of time and used to predict the in vivo effect of such anti-cancer agent on the primary cancer cells in the subject. The ability to make such predictions based on tests of live, primary cancer cells obtained from a solid tumor of a subject was heretofore unknown.

In some embodiments, the tissue samples are dissociated into single cells. The single cells may be primary cancer cells derived from a tissue sample obtained from a subject having cancer. The single cells may also be normal (e.g., non-cancerous cells) derived from a tissue sample obtained from a subject not having cancer or from a tissue sample from a subject having cancer, but the tissue from which the tissue sample is derived does not comprise cancer cells. Dissociating refers to breaking down the extracellular components of a tissue so that single cells remain. Any method known in the art may be used to dissociate tissue samples into single cells including, but not limited to, enzymatic and physical (e.g., manual dissociation) dissociation. In some embodiments, dissociation comprises enzymatic and physical dissociation. Enzymatic dissociation utilizes papain, collagenase, dispase, trypsin, and/or hyaluronidase. Physical dissociation comprises magnetic separation, filtering, crushing and/or extrusion.

In some aspects, the present disclosure provides methods for identifying an anti-cancer reagent. These methods comprise: (a) obtaining a tissue sample comprising primary cancer cells from a subject, (b) dissociating the tissue sample into primary cancer cells; (c) contacting the single primary cancer cells with a reagent; and (d) detecting the mass of the single primary cancer cell contacted with the reagent as it passes through a channel, wherein if the normalized mass of the cell contacted with the reagent is less than a control cell that is not contacted with the reagent, the reagent is an anti-cancer reagent. If the mass of the cell contacted with the anti-cancer reagent is the same or increased compared to the control cell, the reagent is not an anti-cancer reagent, with respect to that cell.

Methods for identifying an anti-cancer reagent may be conducted with primary cancer cells from different cancers. Due to the complex nature of cancer, it is highly probable that one reagent will not be an anti-cancer reagent for every type of cancer tested. This is because it is possible that a reagent will not be an anti-cancer reagent for one type of cancer (e.g., brain cancer), but may be an anti-cancer reagent for another type of cancer (e.g., melanoma). In some embodiments, the methods for identifying an anti-cancer reagent are conducted on 1-100 different cancer types. In some embodiments, the methods for identifying an anti-cancer reagent are conducted on 5-25 different cancer types. In some embodiments, the methods for identifying an anti-cancer reagent are conducted on 10-50 different cancer types.

In some embodiments, the classification of cells is at least 85%-100% accurate at allowing only single cells to be measured compared to manual classification. Manual classification refers to examining the particles in a sample by eye and classifying them. In some embodiments, the classification is at least 85%-95% at allowing only single cells to be measured. In some embodiments, the classification is at least 80%-100% accurate at allowing only single cells to be measured. In some embodiments, the classification is at least 80%, 81%, 82%, 83%, 84%, 85%, 86%, 87%, 88%, 89%, 90%, 91%, 92,%, 93%, 94%, 95%, 96%, 97%, 98%, 99%, or 100% accurate at allowing only single cells to be measured.

In some embodiments, the classification is at least 50%-100% accurate at rejecting cell aggregates and debris from the measurement channel compared to manual classification. In some embodiments, the classification is at least 60%-80% accurate at rejecting cell aggregates and debris from the measurement channel. In some embodiments, the classification is at least 70%-90% accurate at rejecting cell aggregates and debris from the measurement channel. In some embodiments, the classification is at least 50%, 55%, 60%, 65%, 70%, 75%, 80%, 85%, 90%, 95%, or 100% accurate at rejecting cell aggregates and debris from the measurement channel.

Flowing fluid samples across a microfluidic device requires maximum flow rate, while still allowing an assay to proceed accurately. In some embodiments, the single cells of this disclosure are flowed into and through the measurement channel by a process known as active loading. Active loading is the pumping of a fluid sample comprising single cells (e.g., cancer cells, normal cells) across a MBM to maximize the flow rate through the channels while still ensuring that the flow rate is slow enough to accommodate the classification and detection of the mass of single cells. In active loading, the fluid samples comprising the single cells are first classified in a sample channel before single cells are flowed into the measurement channel.

We have characterized the patterns of single cell mass change in response to drugs and radiation therapy in live single cells (including those from primary solid tumors) as a means to rapidly determine the sensitivity of a patient's tumor cells to treatments. Growing and dividing cells generally must increase their mass and dying or growth arrested cells do not have this same requirement and may have no mass increase or lose mass. The baseline mass profile (e.g. increasing=growth, decreasing=dying) biomarker used is the mass accumulation rate (MAR) which can provide key information on single cell biological state which itself can be a biomarker. This can then be compared to treatment ex vivo of cells isolated from the patient and rapidly assessed in minutes to determine whether there is a change in MAR. We have specifically determined responses to MDM2 inhibitors, temozolomide, and radiation and other treatments which each provide distinct MAR profiles as biomarkers and predictors of response to therapy. In addition, we have determined with several agents generalized predictions of effects of agents on MAR that may also be useful in exploring the response of cells to novel targeted therapies for which the mechanisms of action may be unknown or only partially known at the single cell level.

In some aspects, the present disclosure provides methods for evaluating sensitivity of a cancer cell to an anti-cancer reagent. These methods comprise: (a) obtaining a tissue sample comprising primary cancer cells from a subject, (b) dissociating the tissue sample into primary cancer cells; (c) culturing the single primary cancer cells to obtain patient-derived cell lines; (d) contacting the single primary cancer cells with a reagent; (e) engrafting a host subject with the patient-derived cell lines contacted with the anti-cancer reagent; (f) obtaining a tissue sample from the host subject; (g) dissociating the tissue sample from the host subject into single cells; and (h) detecting the mass of the single primary cancer cell contacted with the reagent as it passes through a channel, wherein the mass of the cell contacted with the anti-cancer reagent is compared to the normalized mass of a control cell that is not contacted with an anti-cancer reagent.

In some embodiments, if the mass of the cell contacted with the anti-cancer reagent is decreased compared to the control cell, the cancer cell is sensitive to the anti-cancer reagent. In some embodiments, if the mass of the cell contacted with the anti-cancer reagent is the same or increased compared to the control cell, the cancer cell is resistant to the anti-cancer reagent.

In some embodiments, the single primary cancer cells (and control cells) are contacted with a reagent. In some embodiments, the reagent is an anti-cancer reagent, wherein the reagent is known to kill or inhibit the growth and/or proliferation of at least some cancer cells. Contacting means that the cells are exposed to the reagent (e.g. in culture) for a set period of time. The length of time that cells are contacted with a reagent will vary based on numerous factors including, but not limited to, the stage of the cancer (e.g., I, II, III, or IV), the tissue from which the cell is derived, the reagent that is being contacted, the presence of more than one reagent, and the ability to culture the cell. In some embodiments, contacting is for 0.5-20 days. In some embodiments, contacting is for 5-15 days. In some embodiments, contacting is for 2-10 days. In some embodiments, contacting is for 0.5, 1, 2, 3, 4, 5, 6, 7, 8, 9, 10, 11, 12, 13, 14, 15, 16, 17, 18, 19, or 20 days.

A reagent (e.g., anti-cancer reagent) is a drug that is administered to cells. A drug need not be approved by the FDA to be administered to cells. Non-limiting examples of classes of reagents include small molecules, biologics, cell damaging agents, and oligonucleotides. Small molecules are compounds with a mass of less than 7500 atomic mass units (amu). Small molecules are typically not polymers with repeating units. In certain embodiments, a small molecule has a molecular weight of less than about 1500 g/mol. In certain embodiments, the molecular weight of the polymer is less than about 1000 g/mol. Also, small molecules typically have multiple carbon-carbon bonds and may have multiple stereocenters and functional groups.

Non-limiting examples of small molecules that may be administered to cells include abemaciclib, imatinib (Gleevec), gefitinib (Iressa), erlotinib (Tarceva), sunitinib (sutent), lapatinib (Tykerb), nilotinib (Taigna), sorafenib (Nexavar), temsirolimus (CCI-779), everolimus (afinitor), pazopanib (Votrient), crizotinib (Xalkori), ruxolitinib (jafaki), vandetenib (Caprelsa), axitinib (Inlyta), bosutinib (Bosulif), cabozantinib (Cometriq), ponatinib (Iclusig), regorafenib (Stivagra), ibrutinib (Imbruvica), trametinib (Mekinist), perifosine, bortezomib (Velcade), carfilzomib (Kyprolis), marizomib (NPI-0052), batimastat (BB-94), neovastat (AE-941), prinomastat (AG-3340), rebimistat (BMS-275291), ganetespib, NVP-AUY922, marimastat (BB-2516), obatoclax (GX15-070), and navitoclax (ABT-263).

A biologic is a living organism, substance derived from a living organism, or a laboratory-produced version of a substance derived from a living organism. Non-limiting examples of biologics include immune checkpoint inhibitors, immune cell therapies, therapeutic antibodies, and therapeutic vaccines. Immune checkpoint inhibitors bind and inhibit the activity proteins on the surface of immune cells (e.g., T-cells) that limit the proliferation and/or activity of immune cells. Non-limiting examples of immune checkpoint inhibitors include pembrolizumab (Keytruda), nivolumab (Opdivo), and atezolizumab (Tecentriq). Immune cell therapies collect immune cells from a subject, genomically modify the immune cells so that they attack tumor cells, and re-infuse the immune cells into the subject. Non-limiting examples of immune cell therapies include tisagenlecleucel (Kymriah) and axicabtagene ciloleucel (Yescarta). Therapeutic antibodies are antibodies that are made in the laboratory and bind to target proteins in a subject to treat a disease or condition. Non-limiting examples of therapeutic antibodies include trastuzumab (Herceptin), rituximab (Rituxian), ofatumumab (Azerra), almtuzumab (Campath), ado-trastuzumab emtansine (Kadcyla), brentuximab vedotin (Adcetris), and blinatumomab (Blincyto).

Cell damaging agents are drugs that damage specific regions of cells including, but not limited to, the DNA, mitochondria, cytoskeleton, and/or cell membrane. Non-limiting examples of cell damaging agents include Temozolomide (Temodar), Abraxane, doxorubicin, carboplatin, cyclophosphamide, daunorubucin, epirubicin, 5-fluorouracil, gemcitabine, eribulin, ixabepilone, methotrexate, mitomycin, mitoxantrone, vinorelbine, paclitaxel, docetaxel, thitepa, vincristine, and capecitabine.

In some embodiments, the single cells (e.g., cancer cell, control cell) are exposed to radiation. Radiation is administered to cancer cells to kill or inhibit their growth and/or proliferation. The dose and the type of radiation will vary based on factors including, but not limited to, the type of cancer, the duration of radiation, the presence of other anti-cancer reagents. Non-limiting examples of radiation include X-rays, gamma rays, and charged particles.

“Pharmaceutical agent,” also referred to as a “drug,” or “therapeutic” is used herein to refer to an agent that is administered to a subject to treat a disease, disorder, or other clinically recognized condition that is harmful to the subject, or for prophylactic purposes, and has a clinically significant effect on the body to treat or prevent the disease, disorder, or condition. Therapeutic agents include, without limitation, agents listed in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005.

An oligonucleotide may also be administered to a cell. In some embodiments, the oligonucleotide binds and inhibits the activity of one or more genes in the cells. In some embodiments, the oligonucleotide binds and promotes the activity of one or more genes in the cells. Non-limiting examples of oligonucleotides include double-stranded DNA (dsDNA), single-stranded DNA (ssDNA), double-stranded RNA (dsRNA), single-stranded RNA (ssRNA), short-hairpin RNA (shRNA), short-interfering RNA (siRNA), non-coding RNA (ncRNA), long non-coding RNA (lncRNA), and microRNA (miRNA).

In some embodiments, dissociating the tissue sample into single cells, contacting the single cells with a reagent (e.g., anti-cancer reagent), and detecting the mass of the single cells contacted with the reagent are performed within 1 hour-1 month of obtaining the tissue sample from the subject. In some embodiments, dissociating the tissue sample into single cells, contacting the single cells with a reagent (e.g., anti-cancer reagent), and detecting the mass of the single cells contacted with the reagent are performed with 1 week-1 month of obtaining the tissue sample from the subject. In some embodiments, dissociating the tissue sample into single cells, contacting the single cells with a reagent (e.g., anti-cancer reagent), and detecting the mass of the single cells contacted with the reagent are performed with 1 day-1 week of obtaining the tissue sample from the subject. In some embodiments, dissociating the tissue sample into single cells, contacting the single cells with a reagent (e.g., anti-cancer reagent), and detecting the mass of the single cells contacted with the reagent are performed with 1 hour, 6 hours, 24 hours, 48 hours, 72 hours, 96 hours, 1 week, 1.5 weeks, 2 weeks, 2.5 weeks, 3 weeks, 3.5 weeks, or 1 month of obtaining the tissue sample from the subject.

In some embodiments, the single primary cancer cells (and control cells) are cultured to produce patient-derived cell lines. Patient-derived cell lines may be cultured for short-term (e.g., 1 day-1 week) or long-term (e.g., 1 week-6 months) studies. In some embodiments, the patient-derived cell lines are contacted with a reagent (e.g., anti-cancer reagent) and the mass of single cells in the patient-derived cell lines are detected as they pass through a channel.

In some embodiments, the patient-derived cell lines are administered to a host subject. A host subject is a subject that will be engrafted with the patient-derived cell lines to determine the effect of the reagent on the patient-derived cells. This generates a patient-derived xenograft (PDX). A host subject may be any subject provided herein. In some embodiments, a host subject is a mouse. In some embodiments, the host subject is engrafted with the patient-derived cell lines prior to contacting the single cells with a reagent. In some embodiments, the host subject is engrafted with the patient-derived cell lines after contacting the single cells with a reagent. Single cells can then be isolated from the PDX, and the mass of these single cells can be detected by any methods described herein.

Mass of the cell can be combined with other markers such as mass rate of change, cell surface markers, and other characteristics of the cell in order to more fully characterize the cell or the effect of the therapeutic reagent on the cell. Thus, other cellular parameters may also be detected to determine whether a cancer cell is sensitive to an anti-cancer reagent. Non-limiting examples of such other cellular parameters include mass rate of change, cell surface markers, expression of pro-apoptotic proteins in cells, membrane permeability, mitochondrial membrane permeability, and cell aggregation. In some embodiments, these other cellular parameters are combined with detection of the mass of single cells.

Additional conceptual biomarkers with possible related nature are Mass Cytometry which tags cells typically with antibodies to measures and distinguish differences in them and then uses mass spec to distinguish these cells. This differs in that the intrinsic growth of the cell is not dynamically measured and the assay leads to destruction of the cell by the nature of the measurement and cells cannot be recovered or same single cells studied repeatedly using multiple methods.

The technology also includes use of live time lapse imaging of cells treated in parallel and monitored using imaging based methods combined with cell state fluorescent markers. These imaging based biomarkers add to the mass biomarker to include apoptotic status, live cell status, and cell cycle specifics for individual cells in a population. Integration of this data with the mass biomarker data from the SMR gives a very specific and rapid measurement of cell health after drug treatment which is functional and goes beyond a genomic assessment.

Any appropriate condition or disease of a subject may be evaluated using the methods herein, typically provided that a test agent may be obtained from the subject that has a material property that is indicative of the condition or disease. The condition or disease to be detected may be, for example, a fetal cell condition, HPV infection, or a hematological disorder, such as sickle cell disease, sickle cell trait (SCT), spherocytosis, ovalocytosis, alpha thalassemia, beta thalassemia, delta thalassemia, malaria, anemia, diabetes, leukemia, cancer, infectious disease, HIV, malaria, leishmaniasis, babesiosis, monoclonal gammopathy of undetermined significance or multiple myeloma. Examples of cancers include, but are not limited to, Hodgkin's disease, Non-Hodgkin's lymphoma, Burkitt's lymphoma, anaplastic large cell lymphoma, splenic marginal zone lymphoma, hepatosplenic T-cell lymphoma, angioimmunoblastic T-cell lymphoma (AILT), multiple myeloma, Waldenstram macroglobulinemia, plasmacytoma, acute lymphocytic leukemia (ALL), chronic lymphocytic leukemia (CLL), B cell CLL, acute myelogenous leukemia (AML), chronic myelogenous leukemia (CML), T-cell prolymphocytic leukemia (T-PLL), B-cell prolymphocytic leukemia (B-PLL), chronic neutrophilic leukemia (CNL), hairy cell leukemia (HCL), T-cell large granular lymphocyte leukemia (T-LGL) and aggressive NK-cell leukemia. In one embodiment, the cells are from a subject having or suspected of having sickle cell disease. The foregoing diseases or conditions are not intended to be limiting. It should thus be appreciated that other appropriate diseases or conditions may be evaluated using the methods disclosed herein.

Methods are also provided for testing candidate therapeutic agents for treating a condition or disease in a subject. The methods typically involve: (a) perfusing a fluid comprising one or more cells from the subject through the any of the microfluidic devices, described herein, (b) administering one or more compounds to the fluid of (a), or wherein the fluid comprises the one or more compounds; (c) determining a property of one or more of the cells; and (d) comparing the property to an appropriate standard, wherein the results of the comparison are indicative of the status of the condition or disease in the subject.

The two or more compounds may be administered to the fluid sequentially or simultaneously. An effective therapeutic agent may be identified based on the comparison in (d). The cells may be from a subject, and the effective therapeutic agent may be administered to the subject. The compounds may be from a library of compounds, and in some embodiments, are candidate therapeutic agents.

In some embodiments, a method for analyzing, diagnosing, detecting, or determining the severity of a condition or disease in a subject, includes (a) perfusing a fluid comprising one or more cells from the subject through the any of the microfluidic devices, described herein, (b) determining a property of one or more of the cells; and (c) comparing the property to an appropriate standard, wherein the results of the comparison are indicative of the status of the condition or disease in the subject.

An “appropriate standard” is a parameter, value or level indicative of a known outcome, status or result (e.g., a known disease or condition status). An appropriate standard can be determined (e.g., determined in parallel with a test measurement) or can be pre-existing (e.g., a historical value, etc.). The parameter, value or level may be, for example, a transit characteristic (e.g., transit time), a value representative of a mechanical property, a value representative of a rheological property, etc. The appropriate standard can be a mechanical property such as mass of a cell obtained from a subject who is identified as not having the condition or disease or can be a mechanical property of a cell obtained from a subject who is identified as having the condition or disease.

The magnitude of a difference between a parameter, level or value and an appropriate standard that is indicative of known outcome, status or result may vary. For example, a significant difference that indicates a known outcome, status or result may be detected when the level of a parameter, level or value is at least 1%, at least 5%, at least 10%, at least 25%, at least 50%, at least 100%, at least 250%, at least 500%, or at least 1000% higher, or lower, than the appropriate standard. Similarly, a significant difference may be detected when a parameter, level or value is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the level of the appropriate standard. Significant differences may be identified by using an appropriate statistical test. Tests for statistical significance are well known in the art and are exemplified in Applied Statistics for Engineers and Scientists by Petruccelli, Chen and Nandram Reprint Ed. Prentice Hall (1999).

The methods herein also provide for monitoring and/or determining the effectiveness of a therapeutic agent. One method for monitoring the effectiveness of a therapeutic agent for treating a disease or condition in a subject includes (a) perfusing a fluid comprising one or more cells from the subject through the microfluidic device described above; (b) determining a property of one or more of the cells; (c) treating the subject with the therapeutic agent; and (d) repeating steps (a) and (b) at least once wherein a difference in the property of one or more cells is indicative of the effectiveness of the therapeutic agent.

### EXAMPLES

### Example 1—Experimental Methods

Image Analysis. Live images are acquired using a monochrome camera (BFS-U3-13Y3M-C, FLIR). Custom software coded in LabVIEW 2017 (National Instruments) is used to analyze images in real-time and integrate the image feedback with automated pneumatic control. A standard computer equipped with a 20154-core CPU with 8 Gb of RAM was capable of analyzing at least 60 frames s⁻¹ stably. Settings specific to the image-processing code were calibrated using a suspension of polystyrene beads (Duke Scientific, #4207A) prior to loading biological samples on the serial suspended microchannel resonator (sSMR).

Pneumatic Control. The sSMR features four fluidic ports. These ports connect to pneumatically sealed satellite reservoirs containing media or sample in sterile secondary vials. Independent electronic pressure regulators (QPV1TFEE030CXL, Proportion Air) control the pressure within the reservoir, which drives flow across the sSMR. Regulators are supplied with 5% CO₂ gas, and the microfluidic chip and satellite reservoirs are kept at 37° C. using custom aluminum heat exchangers to maintain incubator-like conditions.

Sample Preparation. All liquids were filtered with 0.2 m filters prior to use in the PDMS device or in cell culture. L1210 (murine lymphocytic leukemia, 87092804-1VL, ECACC/Sigma-Aldrich) and BaF3 (murine pro-B, Riken BioResource Center) cells were cultured in RPMI-1640 with L-glutamine (11875-093, Gibco) with added 10% dialyzed fetal bovine serum (F0392-500 mL, Sigma), 25 mM HEPES (15630-080, Gibco), and 1% ABAM (15240-062, Gibco). Cells are prepared by centrifuging for 5 min at 200×g, removing the supernatant, and resuspension in fresh pre-warmed complete RPMI as defined above. These cell lines were not tested for mycoplasma contamination or authenticated.

Patient-derived cells from six different types of brain tissues were assessed for drug sensitivity in the sSMR: non-tumor brain tissue from epilepsy surgery, glioblastoma, recurrent glioblastoma, breast metastasis, lung metastasis, and primary CNS lymphoma. Resected samples obtained with patient consent to research (Brigham and Women's Hospital, DF/HCC IRB-approved consent protocol 10-417) were enzymatically and physically dissociated using the Brain Tumor Dissociation Kit P (130-095-942, Miltenyi Biotec) and gentleMACS Dissociator (130-093-235, Miltenyi Biotec). Cells were cultured in Neurocult NS-A proliferation media (05702, Stemcell Technologies) containing 20 ng mL⁻¹ epidermal growth factor (130-093-825, Miltenyi Biotec) and 10 ng mL⁻¹ fibroblast growth factor (130-093-564, Miltenyi Biotec).

After at least 48 h in culture (with the exception of CNS lymphoma which was cultured for 24 h), persistent red blood cells were removed with RBC lysis buffer (00-433-57, Thermo Fisher Scientific). The remaining cells were then dissociated with Accutase (A6964, Sigma-Aldrich) and further purified via demyelination (130-096-733, Miltenyi Biotec) with mass spectrometry separation columns (130-042-201, Miltenyi Biotec), or debris removal (130-109-398, Miltenyi Biotec). The purified cells were plated in 6-well or 24-well plates and allowed to recover in the well plate for 48-96 h before addition of the drug. Specific timelines in culture and drugging regimens for each tissue type can be found in Table 1. Prior to loading samples on the sSMR for drug response measurements, cells were dissociated into a single-cell suspension using Accutase and gentle pipetting. Cells were resuspended at a concentration of 100,000 cells mL-1 in Neurocult NS-A (as prepared above) with the same concentration of drug or DMSO as their respective culture.

Device Preparation. The sSMR is cleaned prior to each experiment with 10% bleach for 10 min, followed by a 20-min rinse with DI-H₂O. Persistent biological debris is removed with 0.25% Trypsin-EDTA. After cleaning, the device is passivated with 1 mg mL⁻¹ PLL-g-PEG in H₂O for 10 min at 37° C.

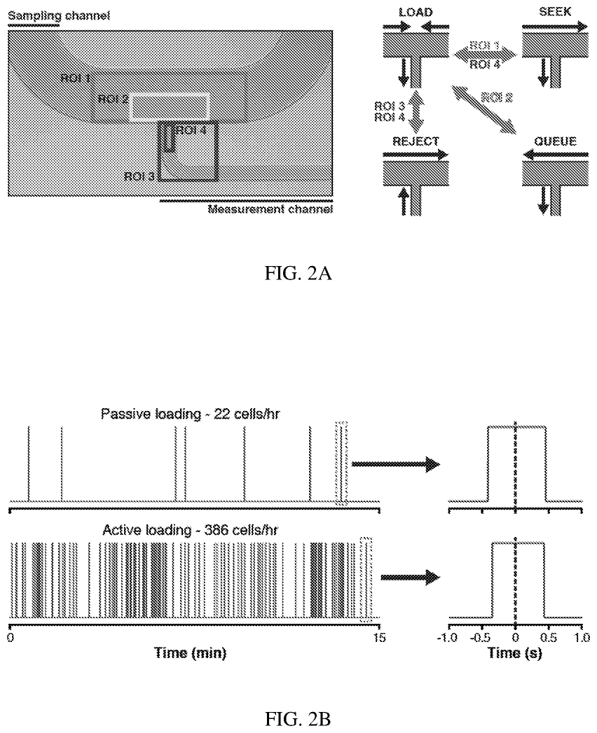

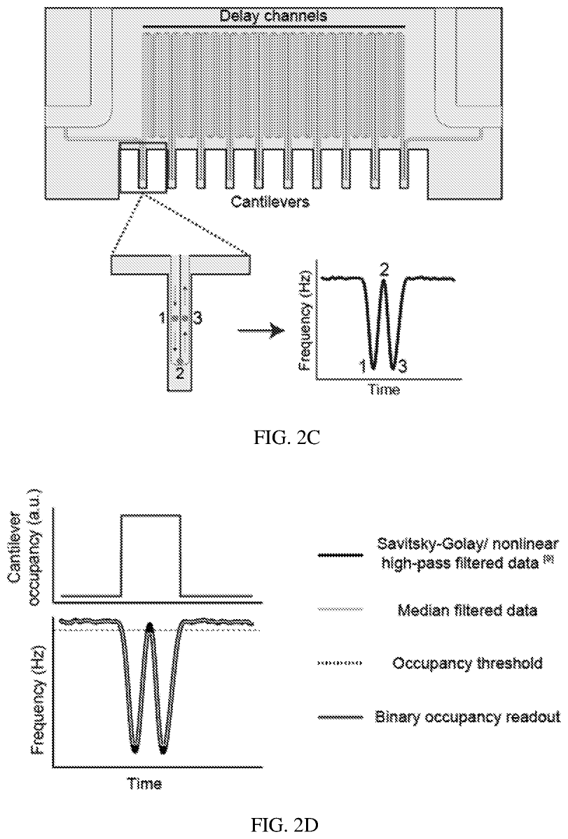

SMR Measurementsfor Transit Time Detection. To detect cells and characterize transit time (e.g., as in FIGS. 2A-2B), resonant frequency data was collected from the first cantilever of a sSMR (FIGS. 2C-2D). Savitsky-Golay and nonlinear high-pass filters were used to isolate mass signals from measurement noise (see, e.g., Cheung, K., Gawad, S. & Renaud, P. Impedance spectroscopy flow cytometry: on-chip label-free cell differentiation. Cytometry. Part A: the journal of the International Society for Analytical Cytology 65, 124-132 (2005)), and subsequent median filtering (frame length of 49) and threshold detection were implemented such that all below-threshold points were set to zero and all above-threshold points were set to one. These filtered data provide a binary characterization of SMR occupancy seeing as the resonant frequency shifts caused by cell transit led to above-threshold measurements. Single-cell transit times were subsequently quantified by determining the number of consecutive above-threshold measurements collected for each cell.

Primary Sample Handling. The six primary samples underwent the same protocol with regards to disassociation, recovery, and drugging; however, the exact timeline of each tissue varied slightly based on the amount of tissue and drug used (Table 1, below). After at least [culture time] in culture (with the exception of CNS lymphoma which was cultured for 24 hours), persistent red blood cells were removed with RBC lysis buffer (00-433-57, Thermo Fisher Scientific). The remaining cells were then dissociated with Accutase (A6964, Sigma-Aldrich) and further purified via demyelination (130-096-733, Miltenyi Biotec) with MS separation columns (130-042-201, Miltenyi Biotec), or debris removal (130-109-398, Miltenyi Biotec). The purified cells were plated in 6 or 24 well plates and allowed to recover in the well plate for [recovery time] before addition of the drug. After [drug duration] days, the samples were prepared for sSMR for drug response measurements by dissociation into a single-cell suspension using Accutase and gentle pipetting. Cells were resuspended at a concentration of 100,000 cells/mL in Neurocult NS-A (as prepared above) with the same concentration of drug or DMSO as their respective culture. Measurements for sample viability were determined (Table 2, below).

TABLE 1
Culture Timeline

| | [culture time] | [recovery time] | [drug duration] | | | |
|---|---|---|---|---|---|---|
| Tissue Type | (days) | (days) | (days) | Vehicle | Drug 1 | Drug 2 |
| Normal brain | 2 | 2 | 3 | DMSO | 250 μM | |
| | | | | | TMZ | |
| Glioblastoma | 5 | 5 | 8 | DMSO | 250 μM | |
| | | | | | TMZ | |
| Recurrent | 2 | 4 | 3 | DMSO | 1 μM | |
| Glioblastoma | | | | | Abema | |
| Breast Met | 3 | 4 | 3 | DMSO | 1 nM | 100 nM |
| | | | | | RAD | Abema |
| Lung Met | 3 | 5 | 3 | Water | 100 μM | |
| | | | | | Carbo | |
| CNS | 1 | 1 | 2 | DMSO | 10 nM | |
| Lymphoma | | | | | Ibrutinib | |

TABLE 2
Sample Viability

| | Vehicle | Drug 1 | Drug 2 |
|---|---|---|---|
| | Viability | Viability | Viability |
| Tissue Type | (Live/Dead) | (Live/Dead) | (Live/Dead) |
| Normal brain | 35% | 30% | |
| Glioblastoma | 73% | 69% | |
| Recurrent | 67% | 59% | |
| Glioblastoma | | | |
| Breast Met | 35% | 32% | 35% |
| Lung Met | 80% | 74% | |
| CNS Lymphoma | 74% | 65% | |

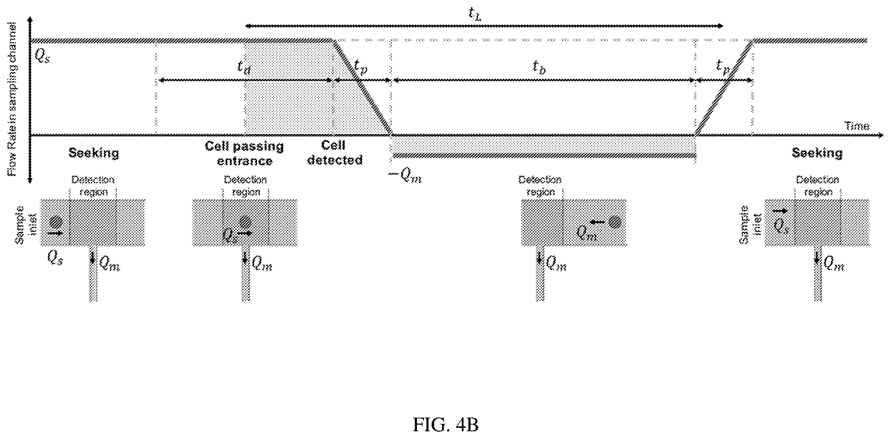

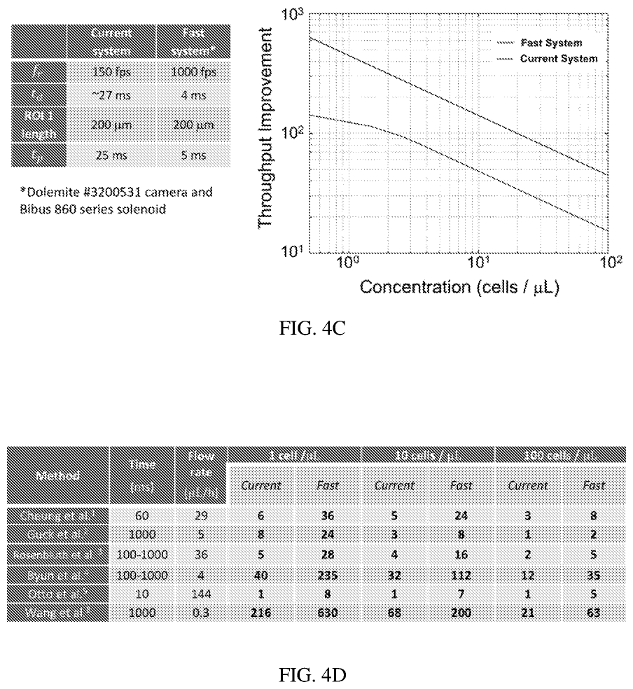

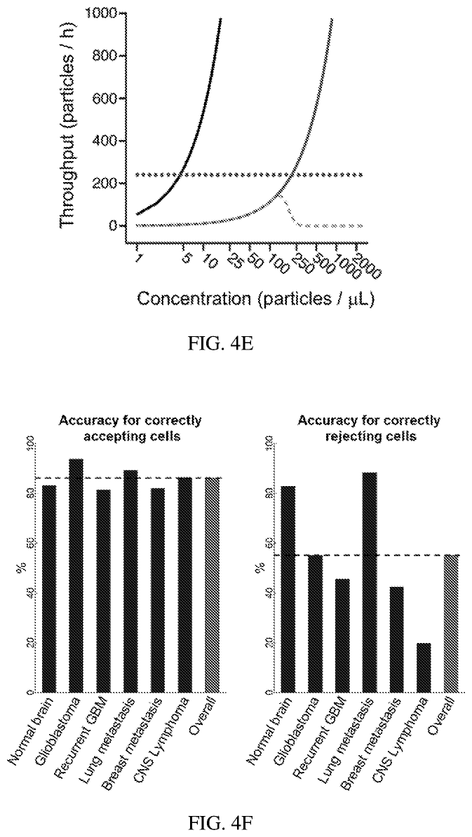

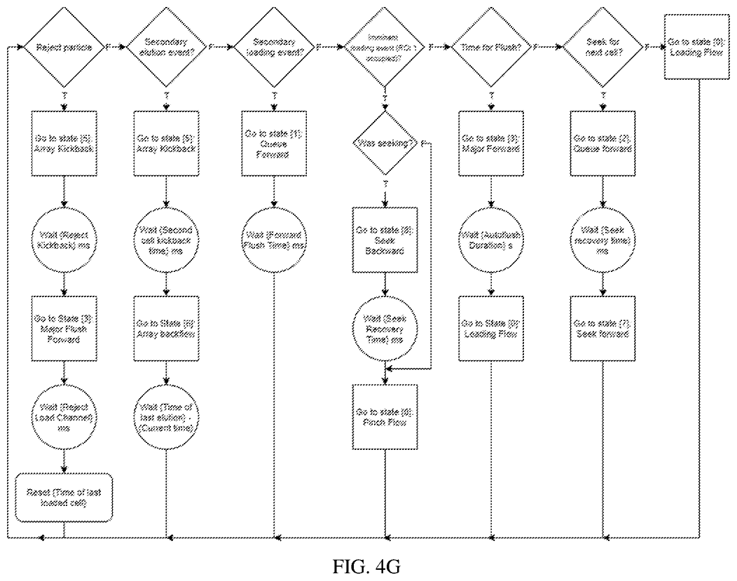

Automated particle detection. FIG. 4A shows an example of automated particle classification. Panels (I) through (IV) depict examples of particles automatically classified as a “singlet” (I), “doublet” (II), “multiple singlet” (III), and “debris” (IV). Panel (V) is a particle classification diagram depicting the automated particle classification logic. The background image is created by calculating the median value for each pixel from the past X frames, where X is a user designated control. The present frame is subtracted from the median image, effectively leaving behind an image showing only objects in motion. A user inputted pixel threshold is subtracted from the subtracted image, and the resultant values are coerced to a value between 0 and 255. The ‘AutoBinaryThreshold’ subVI is used to transform this image into a binary image, with pixel values of 0 or 1. Morphology of the resultant image is smoothed with automedian, dilate, convex hull, and hole filling subVIs. The ‘Particle Analysis Report’ subVI then identifies continuous pixel regions with a value of 1, and generates a list of these particles. Any particle outside of a user determined size (number of pixels) threshold is removed from the list. If there are no particles remaining, the triggering event is determined to have been ‘Debris’. If there are more than one particles within the size threshold then the triggering event is determined to have been ‘Multiple Singlets’. If only one particle is within the size threshold then the X:Y ratio of the bounding rectangle is used to determine whether the particle is a doublet. Particles with an X:Y ratio below the user designated threshold and above the reciprocal of the threshold are considered to be ‘Singlets’. Particles with an X:Y ratio above the user designated threshold or below the reciprocal of the threshold are determined to be ‘Doublets’.

Throughput enhancement provided by Active Loading. The throughput improvements that could be achieved by implementing active loading were evaluated for various single-cell applications that have been described previously in the literature. For this purpose, the improvement metric was defined as the ratio between the effective sampling flow rate and the flow rate that would have been achieved in the measurement channel without active loading. As described below, several assumptions are made in order to estimate the effects of detection and pneumatic control delay in the sampling channel and the ratio of cross sections of the measurement and sampling channels.

Since each detection event during the ‘seek’ operation triggers a loading cycle, the throughput with active loading is a function of cell concentration in the sample. Within the non-zero time frame of the loading cycle, the seeking flow is stopped, reducing the effective sampling flow rate (Qₜ). The effective flow rate is defined by Equation (1):

$${Q}_{t} = \frac{V}{{T}_{t}} ,$$

where V and Tₜ are the total sample volume to be measured and total duration of sampling, respectively.

Assuming a time frame of t_{L} is required to load each cell into the measurement channel from the moment of detection, one can calculate the total measurement duration (Tₜ) as a function of cell concentration (C) as follows in Equation (2):

Tₜ=Tₛ+CVt_{L},

where Tₛ is the total time required to flow the same sample of volume V at a flow rate of Qₛ with no particle-detection. Inserting Equation (2) into (1) provides Equation (3):

$${Q}_{t} = \frac{V}{( {T}_{s} + {CVt}_{L} )} = \frac{1}{\frac{1}{{Q}_{s}} + {t}_{L} C} ,$$

Equation (3) is a general equation defining the effective flow rate provided by active loading, when the detection and loading events are taken into account. The time required to load each cell into the measurement channel is modeled assuming non-ideal system components with non-zero time responses. In FIG. 4B, the change of flow rate in the sampling channel is illustrated as a function of time during a cell loading cycle. The loading cycle starts when a cell is detected in the sampling channel as it is flowing at a seeking flowrate of Qₛ. The latency due to the pneumatic instrumentation and the detection scheme cause a detected cell to miss the entrance of the measurement channel, creating an excess volume (shaded) to be sampled into the measurement channel before the detected particle. For simplicity, two fundamental time delays dictated by the detection time (t_{d}) and pneumatic latency (tₚ) are defined. It is assumed that before the cell enters the measurement channel, all excess volume is loaded into the measurement channel at a flow rate of Qₘ, which determines the time required to back flow a cell into the measurement channel (t_{b}). Since the sampling into the measurement channel is from downstream only, the detection region is centered at the channel entrance, and the pneumatic response is linear in time, one can approximate the loading time of a detected cell as Equation (4):

$${t}_{L} = \frac{{t}_{d}}{2} + \frac{3 {t}_{p}}{2} + ( \frac{{t}_{d}}{2} + \frac{{t}_{p}}{2} ) \frac{{Q}_{s}}{{Q}_{m}} = \frac{{t}_{d} + 3 {t}_{p}}{2} + \frac{( {t}_{d} + {t}_{p} ) {Q}_{s}}{2 {Q}_{m}} .$$

Here, Qₘ is the flow rate in the measurement channel and inversely proportional to the measurement time required for the targeted application (or proportional to the measurement bandwidth) and kept constant at all times during the seeking and loading cycles. For the purpose of this analysis, it was assumed that the backflow rate is identical to the measurement flow rate. However, faster rates could be utilized with more complicated control algorithms, which would require the replacement of Qₘ in Equation (4) above. As the merit of active loading relies on achieving Qₛ>>Qₘ, Equation (4) simplifies to Equation (5):

$${t}_{L} \approx \frac{( {t}_{d} + {t}_{p} ) {Q}_{s}}{2 {Q}_{m}} .$$

Using Equations (3) and (5), the net improvement of active loading as a function of system and sample variables is calculated according-to Equation (6):

$$\frac{{Q}_{t}}{{Q}_{m}} = \frac{1}{\frac{{Q}_{m}}{{Q}_{s}} + \frac{( {t}_{d} + {t}_{p} ) {CQ}_{s}}{2}} .$$

Equation (6) shows that the throughput improvement for a given sample concentration is a function of the seeking flow rate. Due to the non-zero time response of the detector and pneumatics, the seeking flow rate has an optimal value to achieve the maximum throughput improvement for a given cell concentration. This optimal rate (Q′ₛ) is calculated as a function of system variables, sample concentration and measurement flow rate requirement by taking the derivative of Equation (6), equating it to zero and solving for Qₛ by Equation (7):

$${Q}_{s}^{′} = \sqrt{\frac{2 {Q}_{m}}{( {t}_{d} + {t}_{p} ) C}} .$$

Finally, the throughput improvement from active loading at the optimal seeking flow rate is calculated by inserting Equation (7) into (6) to arrive at Equation (8):

$$\frac{{Q}_{t}}{{Q}_{m}} {\vert}_{{Q}_{s} = {Q}_{s}^{′}} = \frac{1}{\sqrt{2 ( {t}_{d} + {t}_{p} ) {CQ}_{m}}} .$$

Equation (8) demonstrates that the benefit of active loading increases for samples that are low in concentration, for applications where a slow measurement flow rate is necessary and for measurement systems with low latency.

In the equations above, t_{d} is defined by the method utilized for detecting cells in the sampling channel. Although faster detection methods such as electrical, capacitive, interferometric could be utilized here, detection by imaging was focused on, as it provides additional benefits for active loading (e.g., debris rejection, cell shape determination, fluorescence measurements). For the special case of the optical detection using a camera, if one conservatively assumes that 4 frames are necessary to successfully detect a cell at the shutter speed of the camera, setting t_{d}=4/fᵣ. Therefore, the frame rate and field of view puts an upper bound on Qₛ. Using Equations (6-8), a plot was generated (FIG. 4C) for the throughput improvement for a range of sample concentrations for the system used herein (Current System) and for a system with the same channel dimensions but faster detection and pneumatic control (Fast System). For these two scenarios, the specifications listed in the table of FIG. 4C were used. The size of the detection region was assumed to be centered around the measurement channel entrance and 200 microns long. Therefore, a camera that has a faster shutter speed would enable faster seeking flow rates, increasing the throughput improvement for samples with low concentration of cells.

The plot of FIG. 4C shows that the throughput improvement is a strong function of sample concentration and that a more than 100-fold improvement is theoretically possible for low concentration samples. Although the benefit of active loading drops for samples that are concentrated, fast pneumatics and detection schemes could still enable a more than 10-fold improvement over traditional methods.

Finally, the extent to which other single-cell microfluidic sensors could benefit from the active loading approach was determined. FIG. 4D shows estimated theoretical throughput improvements possible with active loading if applied to various single-cell measurement techniques. For conducting a fair comparison, it was assumed that the same flow speed that was used in the corresponding reference is achieved in the measurement channel utilized herein. The optimal seeking flow rate was calculated for the current system and a fast system. In the event that the optimal seeking flow rate exceeded what is achievable with the sampling channel camera, the maximum achievable flow rate was used instead.

Throughput modeling with desired minimum particle spacing. The throughput achievable by passively loading cells into a sSMR chip is Poisson limited. The average throughput (F_{Passive}) is equal to the concentration (C) of cells in the sample multiplied by the volumetric flow rate (Q_{V}) through the chip, where V is the total chip volume and T is the total time required for a cell to travel through the entire chip, according to Equation (9):

$${F}_{Passive} = {CQ}_{V} = C \frac{V}{T} .$$

The precision of mass accumulation rate measurements made by a sSMR array is inversely proportional to T (see, e.g., Cermak, N. et al. High-throughput measurement of single-cell growth rates using serial microfluidic mass sensor arrays. Nat Biotech 34, 1052-1059 (2016)). Therefore, to achieve a biologically relevant measurement precision, the volumetric flow rate through the sSMR chip was kept constant such that, on average, cells travel through the chip in ˜15 minutes. A constant volumetric flow rate (Q_{V}) in Equation (9) results in a concentration-limited throughput. The sSMR devices utilized for mammalian cells have a volume of 0.283 μL, resulting in a volumetric flow rate of approximately 1.132 μL/h. For this case, Equation (9) simplifies to Equation (10):

F_{Passive}=1.132 μL/h×C,

which is plotted as the right-most solid line in the plot shown in FIG. 4E.

Equation (10) represents an idealized case where all of the cells flow at an identical velocity in the measurement channel. Since measuring MAR of a cell requires a set of mass measurements performed by different sensors in the sSMR chip to be assigned to the same cell, variations of cell order in the measurement channel could create discrepancies during this matching process. Cells or particles in the measurement channel have varied velocities that depend on their size and position in the channel. Interaction of cells with channel walls exacerbates this problem by slowing certain cells in the stream. Furthermore, doublet formation in the measurement channel, or from simultaneously loading collisions in high concentration samples, results in clogging. To address these limitations, a minimum time gap of 15 seconds between events to prevent most collisions and changes in cell order was empirically determined. The average time difference between each cell loading event, (t̅_̅{̅Δ̅}̅) can be calculated by Equation (11):

$$\overline{{t}_{Δ}} = \frac{1}{{F}_{Passive}} .$$

A Poisson probability distribution for time between each loading event can be calculated using Equation (11), which is used to find the fraction of events with a greater-than 15 second spacing for any given concentration. Equation (12):

$$P ( t \geq 15 s ) = 1 - \frac{{e}^{- λ} {λ}^{k}}{k !} = 1 - \frac{( {e}^{- \overline{{t}_{Δ}}} ) ( {\overline{{t}_{Δ}}}^{t} )}{t !} .$$

The effective rate of particles (F_{eff}) is defined as the rate of particles with a time gap of at least 15 seconds between the leading and trailing particle. F_{eff} is thus calculated as the rate of particles entering the array multiplied by the probability of a time gap greater than 15 seconds squared (dashed line in plot shown in FIG. 4E), as in Equation (13):

F_{eff}=(C×1.132 μL/h)×P(t≥15 s)².

The maximum theoretical active loading throughput would be achieved with instantaneous detection and loading from the sampling channel. The maximum throughput would then be divided into a ‘seek’ limited fraction and a ‘queue’ limited fraction. The seek limited throughput limit can be calculated by using Equation (9) and substituting the seeking volumetric rate for the device volumetric rate (plotted as the left-most solid line in the plot shown in FIG. 4E). Equation (14):

F_{active}=54 μL/h×C.

To calculate this ‘queue’ limited portion of active loading, a time delay of 15 seconds that minimizes matching failure was assumed. The throughput in this case is calculated by assuming a uniform loading every 15 seconds (dotted line in plot shown in FIG. 4E). Equation (15):

$${F}_{active} = \frac{1}{{t}_{gap}} = \frac{1}{15} cells \text{/} s = 240 cells \text{/} h .$$

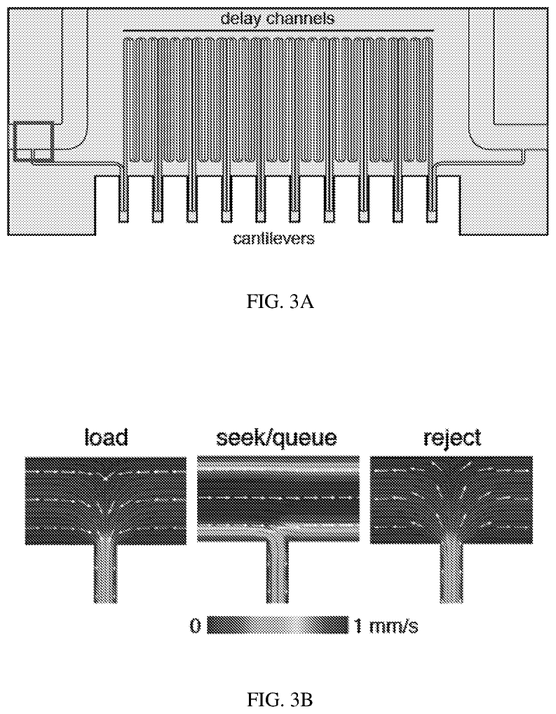

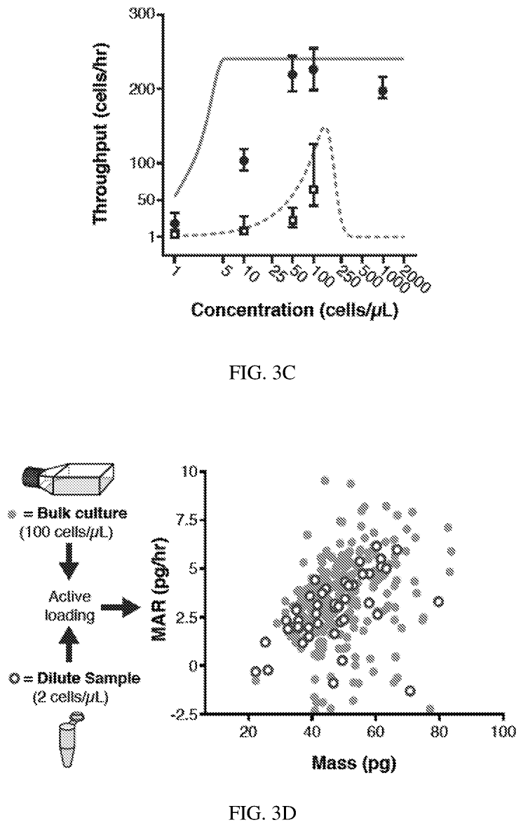

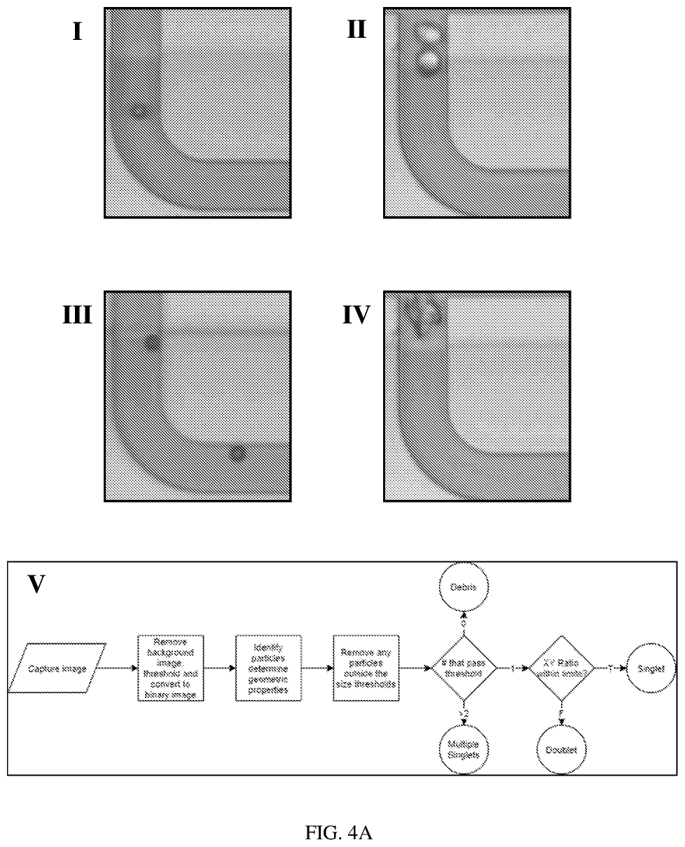

The theoretical throughput curve presented in FIG. 3C is constructed by taking the minimum throughput of either the ‘seek’ or ‘queue’ limited conditions for a particular concentration. As seen in FIGS. 3A-3D, the experimental throughput of the sSMR achieved with active loading does not match this theoretical maximum, particularly for low-concentration samples. This discrepancy is due to the practical throughput limitations imposed by the system's optical and pneumatic components described above.

Accuracy of the real-time cell classification used for active loading. The accuracy of active loading for correctly allowing cells into the measurement channel based on user-specified criteria of the brightfield images that are acquired as cells transition from the sampling channel into the measurement channel was evaluated. Each image was analyzed in real-time by Labview code in order to assess whether or not the particle should be allowed into the measurement channel (accepted) or removed via the sampling channel (rejected). After the experiment, each image was evaluated manually to determine if the real-time decision based on the automated image analysis was correct. User-specified criteria were designed to reject particles that were classified as ‘Doublet’, ‘Multiple Singlets’, or ‘Debris’. When combining all six samples together, the accuracy for correctly allowing particles into the measurement channel was 86% (2040 particles were allowed by the real-time code, 1757 of them were manually classified as single cells) and 55% for correctly rejecting particles (4159 particles rejected by the real-time code, 2295 of them were manually classified as rejection events). The accuracy for each sample is shown in FIG. 4F, which is a plot of the percentage of real-time classifications that are in agreement with the manual validation.

For this application, user-specified settings are typically weighted to avoid rejection criteria. Consequently, this approach tolerates higher rates of single-cell rejection, despite the fact these events should have been accepted. Rejection of single cells is not particularly detrimental to throughput because the seeking code is capable of quickly finding a second event to load into the array, and lowers the probability that debris or clumps of cells may interfere with flow in the measurement channel. Furthermore, the rejected events are recovered in the downstream collection tube, and for situations were sample is limited, the tube could be reloaded back into the system. In some cases, vibration of the instrument from nearby disturbances triggered the acquisition of an image that did not contain a particle. These events, which were not detrimental to the experiment, were not included in the accuracy assessment.

### Example 2—Experimental Design

The high level of control offered by microfluidic devices has proven to be valuable for single-cell biological assay development, where measurement of individual cells or small clusters of cells can now be performed with exquisite fidelity. However, for platforms that incorporate on-chip detection, flow rate is governed by the bandwidth required for the measurement, which imposes limitations on the maximum achievable throughput. Although measurements such as fluorescent intensity or light scattering can approach 10⁵ cells s⁻¹, biophysical methods such as spectroscopy, deformability, and electrical impedance typically require bandwidths in the 0.1 Hz to 10 kHz range, limiting throughput to the range of 1-10,000 cells min⁻¹ (Table 3, below). Throughput for these approaches can be raised by increasing concentration; however, there are often biological and logistical factors that determine the range of achievable sample concentrations. For example, samples processed from primary tissue sources-including biopsies, fine-needle aspirates, blood samples, patient-derived xenograft tissues, and so on-often yield a limited number of cells of interest that set inherent limits on the maximum achievable sample concentration. Additionally, the loading period of particles into a device is limited by Poisson statistics and flow rate, which makes dilute samples especially challenging without increasing flow rate and sacrificing bandwidth.

To decouple this fundamental trade-off between flow rate into the device and measurement bandwidth, an approach called “active loading” was developed where a triggering detector selectively isolates particles from a large, two-port sampling channel into a second smaller measurement channel. Since the flow rates in each channel can be independently controlled, it is possible to set the flow rate in the measurement channel based on the desired measurement bandwidth while dynamically controlling the sampling channel flow rate in order to deterministically load particles into the measurement channel. Using bright-field microscopy as the triggering detector and standard pressure-driven fluidic control components, the throughput for a particle concentration of 50 μL⁻¹ was improved by over 10-fold without changing the measurement bandwidth. By applying active loading to the serial suspended microchannel resonator (sSMR), it was found that buoyant mass and growth properties can be measured from a dilute concentration of only a few cells per microliter in 3 h. In contrast, the same number of measurements would take over 3 days of continuous passive sampling. A key advantage of active loading with imaging is that debris can be rejected in order to reduce clogging and eliminate unnecessary measurement time. This capability was demonstrated by measuring the drug sensitivity from a range of clinical brain tissue and tumor resection samples containing a complex mixture of confounding biological debris after cell purification.

TABLE 3
Measurement bandwidths from microfluidic sensors

| | Measurement | Measurement | |
|---|---|---|---|
| Type of detector | approach | time (ms) | Reference |
| Electrical | Impedance | 60 | Cheung, K., Gawad, S. & Renaud, P. |
| | spectroscopy | | Impedance spectroscopy flow cytometry: |
| | | | on-chip label-free cell differentiation. |
| | | | Cytometry. Part A: the journal of the |
| | | | International Society for Analytical |
| | | | Cytology 65, 124-132 (2005). |
| Mechanical | Optical stretching | 1,000 | Guck, J. et al. The optical stretcher: a |
| | | | novel laser tool to micromanipulate cells. |
| | | | Biophysical journal 81, 767-784 (2001). |
| | Solid constriction | 100 to 1,000 | Rosenbluth, M. J., Lam, W. A. & Fletcher, |
| | (optical readout) | | D. A. Analyzing cell mechanics in |
| | | | hematologic diseases with microfluidic |
| | | | biophysical flow cytometry. Lab on a chip |
| | | | 8, 1062-1070 (2008). |
| | Solid constriction | 100 to 1,000 | Byun, S. et al. Characterizing |
| | (mass readout) | | deformability and surface friction of |
| | | | cancer cells. Proceedings of the National |
| | | | Academy of Sciences of the United States |
| | | | of America 110, 7580-7585 (2013). |
| | Hydrodynamic | 10 | Otto, O. et al. Real-time deformability |
| | constriction | | cytometry: on-the-fly cell mechanical |
| | | | phenotyping. Nature methods 12, 199-202, |
| | | | 194 p following 202 (2015). |
| | Hydrodynamic | 0.1 | Gossett, D. R. et al. Hydrodynamic |
| | stretching | | stretching of single cells for large |
| | | | population mechanical phenotyping. |
| | | | Proceedings of the National Academy of |
| | | | Sciences of the United States of America |
| | | | 109, 7630-7635 (2012). |
| Optical | Image cytometry | 10 | George, T. C. et al. Distinguishing modes |
| | | | of cell death using the ImageStream |
| | | | multispectral imaging flow cytometer. |
| | | | Cytometry. Part A: the journal of the |
| | | | International Society for Analytical |
| | | | Cytology 59, 237-245 (2004). |
| | Image cytometry | 100 to 1,000 | Wang, X. et al. Enhanced cell sorting and |
| | | | manipulation with combined optical |
| | | | tweezer and microfluidic chip |
| | | | technologies. Lab on a chip 11, 3656-3662 |
| | | | (2011). |
| | Raman | 10,000 | Dochow, S. et al. Tumour cell |
| | spectroscopy | | identification by means of Raman |
| | | | spectroscopy in combination with optical |
| | | | traps and microfluidic environments. Lab |
| | | | on a chip 11, 1484-1490 (2011). |

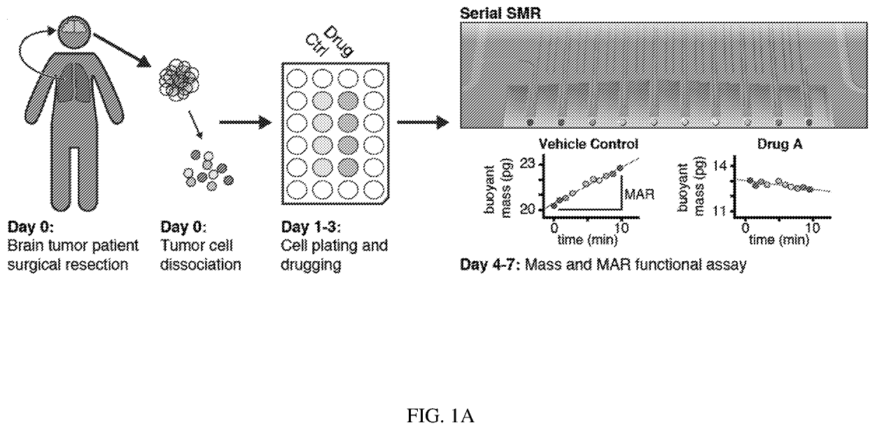

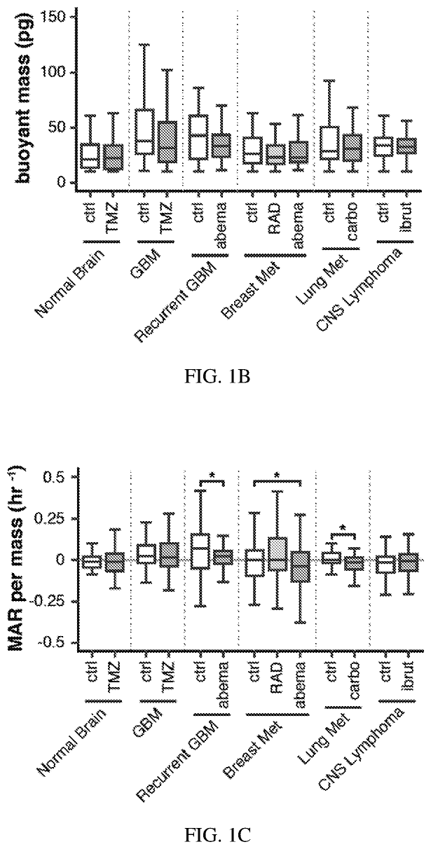

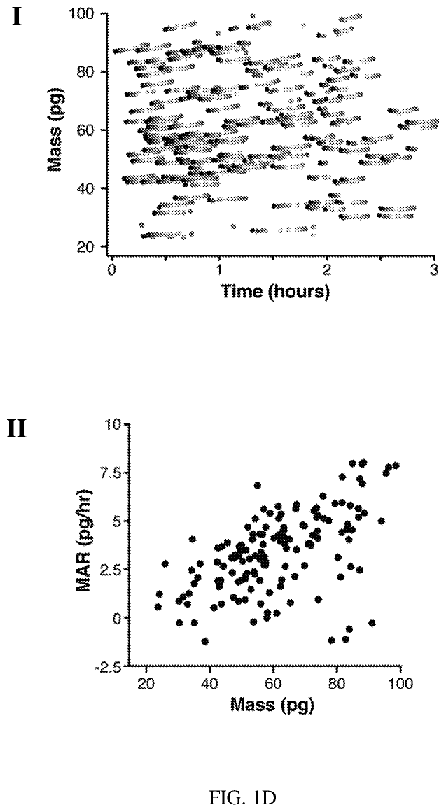

Although numerous methods exist for tissue dissociation and pre-enrichment (e.g., centrifugation, filtration, and magnetic-activated cell sorting (MACS)), they often yield imperfect sample purification by leaving behind significant biological debris or cellular aggregates that make it challenging to analyze or manipulate single cells within microfluidics. The active loading approach described herein improves throughput of single-cell assays by reducing clogging events from debris or aggregates and circumventing limitations imposed by Poisson statistics for loading cells into the measurement channel. For the preclinical studies shown in FIGS. 1A-1C, MACS-based cell enrichment and debris depletion was utilized upstream of the sSMR assay, and it was determined that these samples were still not easily measured without real-time debris rejection enabled by active loading. Thus, active loading is intended to supplement these existing purification methods to enable live-cell measurements from minimally processed and low-input clinical samples. Although the sSMR was used here, active loading could be used to improve performance of other single-cell measurement platforms provided that optical hardware required for imaging can be accommodated. However, benefits from circumventing limitations imposed by Poisson statistics only become meaningful when the necessary measurement time is more than ˜100 ms, which is often the case for biophysical measurements (e.g., as described in Example 1).

While the implementation described here utilizes bright-field imaging with a low-cost camera for label-free detection, further iterations of active loading could achieve higher throughput by triggering with faster cameras or utilize fluorescent intensity readout with a photo-multiplier tube (e.g., as described in Example 1). Additionally, beyond basic geometry-based particle identification used here, improved image processing algorithms may be used to generate more stringent classification criteria to better exclude debris and isolate cells of interest. Given the rapidly increasing number of microfluidic devices and single-cell assays in development for medical use, these universal improvements should be a benefit to the broader community.

### Example 3—Active Loading

Multiple regions of interest (ROIs) are used to detect particles within either the sampling or measurement channels to enable optically triggered activation of various fluidic “states” and isolate individual cells with a defined loading duty cycle (FIGS. 2A, 3B; FIG. 4G and Table 4). The baseline state of the system is a “load” state, which is functionally equivalent to the passive fluidic approach, where the upstream and downstream pressures applied to the sampling channel are equal and a fixed pressure drop is maintained across the measurement channel, thereby setting the average transit time (and the required minimum bandwidth) for individual particles across the detector within the measurement channel. In this state, the volumetric flow into the sampling channel is identical to the flow in the measurement channel and therefore particles are loaded into the measurement channel in a strictly concentration-dependent manner governed by Poisson statistics.

TABLE 4
Complete description of each function triggered by ROIs

| State Name | Description |
|---|---|
| [0] ‘Loading flow’ | Sampling channel upstream and downstream pressures are equal |
| [1] ‘Queue forward’ | Sampling channel upstream pressure is slightly higher than downstream |
| | pressure but nodal pressure at measurement channel entrance remains the |
| | same as [0] |
| [2] ‘Queue backward’ | Same as [1], with reversed sampling flow direction (downstream pressure |
| | higher than upstream) |
| [3] ‘Major forward’ | Sampling channel upstream (cell sample reservoir) pressure is significantly |
| | higher than downstream pressure, but nodal pressure remains the same as [0] |
| [4]‘Major backward’ | Same as [3], with reversed sampling channel flow direction |
| [5] ‘Array kickback’ | Significant flow reversal in the measurement channel such that particles in |
| | the measurement array backflow towards the loading bypass |
| [6] ‘Array backflow’ | Minor flow reversal in the measurement channel |
| [7] ‘Seek forward’ | Sampling channel upstream pressure is moderately higher than downstream |
| | pressure, but nodal pressure remains the same as [0] |
| [8] ‘Seek backward’ | Same as [7], with reversed sampling channel flow direction |

In order to rapidly isolate particles from a dilute sample, the system toggles to a “seek” state. For this task, a pressure drop is applied along the sampling channel to induce a larger volumetric flow rate. During this adjustment, the pressure drop along the measurement channel is unchanged in order to maintain a constant flow rate to ensure consistent single-particle transit time through the detector. The flow along the sampling channel continues until a particle is detected in ROI 1, at which point the system switches to the “load” state to capture the particle in the measurement channel. Since the sampling channel and measurement channel flow rates are largely decoupled, the maximum sampling channel flow rate is limited by the frame rate of the camera used for detection (e.g., as described in Example 1).

To maximize throughput, it is important to identify the next particle available to be measured. To achieve this, the user sets a loading duty cycle that maximizes loading throughput while maintaining the desired measurement bandwidth. Once a particle has entered the measurement channel (as detected by ROI 4), the system repeats the “seek” function. However, the next particle may be detected by ROI 1 prior to completion of the defined loading duty cycle. This occurs for dilute samples where the next particle is not immediately available but is found quickly by the “seek” function as well as high-concentration samples where multiple particles may be proximal to the measurement channel. In order to ensure that no more than one particle is loaded per duty cycle, the system adopts a “queue” state when a cell reaches ROI 2, but the loading duty cycle is not yet complete. The “queue” state is characterized by a brief flush of the particle upstream by introducing a pressure drop along the sampling channel, at which point the system returns to the “load” state. This function repeats as necessary to keep the particle proximal to the measurement channel entrance until sufficient time has elapsed, at which point it is immediately loaded into the measurement channel. This “queue” state, combined with detection in seek mode, is key to enabling high throughput with evenly spaced particle sampling that is not reliant on Poisson statistics.

Finally, to determine if a particle loaded into the measurement channel is a particle of interest and not debris that should be excluded from measurement, the system implements a function driven by real-time image processing. This process relies on user-defined thresholds for particle parameters such as cross-sectional area and x-y ratio (FIG. 4A).

When a particle is loaded into the measurement channel, as detected by ROI 4, ROI 3 captures a bright-field image that is assessed for these parameters. If an undesired particle is loaded, a “reject” state is enabled whereby the pressure drop along the measurement channel is briefly reversed in order to remove the particle. At the same time, a pressure drop is induced along the sampling channel to flush this particle downstream and ensure that it is not recaptured for measurement. This feature allows for the rejection of debris loading events that would otherwise lead to failed measurements and enables high-fidelity measurements on samples with prohibitive amounts of biological debris or aggregates.

To demonstrate active loading, the first mass sensor of an sSMR was used to measure transit time of a murine lymphocytic leukemia cell line (L1210) at a concentration of 50 μL⁻¹ (FIG. 2B, FIGS. 2C-2D, Example 1). For passive loading, only 22 cells h⁻¹ were measured for a desired transit time of 800 ms, while for active loading, 386 particles h⁻¹ were measured without altering the transit time.

### Example 4—Seek, Queue Functions Increase Concentration Dynamic Range

To demonstrate the benefits of active loading for a single-cell assay, it was applied to the sSMR for measuring mass accumulation rate (MAR). The sSMR is well suited for active loading since the sensor transit time is slow (typically ˜600-800 ms) and coincidence within the long (˜50 cm) measurement channel limits the maximum sample concentration (FIGS. 3A-3D). The theoretical ranges of the concentration-dependent throughput for the sSMR with active and passive fluidic implementations were determined (e.g., as described in Example 1). For passive loading, throughput increases for higher concentration samples before reaching a maximum theoretical throughput at an optimal cell concentration. Above this concentration threshold-which is defined by the minimum time required between cells flowing through the sSMR-cell matching failures begin to occur more frequently and the measurement throughput decreases. When this limitation is included, the active loading scheme displays a higher theoretical measurement throughput across all sample concentrations. For dilute-cell samples, this throughput advantage is driven largely by the “seek” function, whereas for medium and high-concentration samples it is driven largely by the “queue” functionality, which ensures sufficient spacing between cells to maintain cell matching fidelity and prevent co-occupancy of the measurement sensors.

These theoretical throughput improvements were tested experimentally by collecting single-cell MAR measurements for L1210 cells seeded at various concentrations (FIG. 3C). For high-concentration samples (above ˜50 cells μL⁻¹), the system was found to perform near the theoretical maximum throughput. For samples of moderate concentration, the advantage of active loading is particularly pronounced: for a sample concentration of 10 cells μL⁻¹, the throughput increased from eight cells per hour for passive fluidic loading to ˜100 cells h⁻¹ using active loading.

To demonstrate the utility of the cell-seeking functionality, single-cell MAR measurements were collected for a sample containing approximately 100 L1210 cells in 50 μL of media (2 cells μL⁻¹) (FIG. 3D). Over the course of a 3-hour experiment, 47 of these cells were isolated for measurement, a data set that would have required approximately 21 hours to collect with passive loading. Furthermore, the fluidic manipulation necessary to conduct this cell-seeking routine did not appear to introduce excessive stress on the cells as there were no significant differences in mass or MAR measurements observed as compared to L1210 cells measured with passive loading (FIG. 3D). In an analogous experiment using a 100 μL sample with approximately 270 hematopoietic cells (2.7 cells μL¹) from a murine pro-B cell line (BaF3), 165 MAR measurements were collected over 3 hours (FIG. 1D). With passive loading, this experiment would have taken >3 days, which would have impacted cell growth dynamics, emphasizing the relevance of substantial throughput gains that are possible with active loading for devices where sampling and measurement flow rates are constrained.

Despite orders of magnitude throughput improvements demonstrated for dilute-cell samples, the throughput did not reach the theoretical limit depicted in FIG. 3C. This is due to nonzero response times of the pneumatic controls, which occasionally causes a cell detected in ROI 1 (FIG. 2A) to overshoot the measurement channel entrance. This overshoot is corrected with a brief flow reversal in the sampling channel, a process that slightly increases the average time between cell loading events (e.g., as described in Example 1).

### Example 5—Rejection Function Reduces Clogging from Debris

A number of confounding factors present challenges to microfluidic technologies in the analysis of single cells from heterogeneous patient biopsy samples. First, the number of cells that one can isolate from samples is highly variable, and often limited by either the biopsy sample size or isolation protocols. Additionally, primary samples generally present with a high level of biological debris and particulate aggregation, which limit flow rate by clogging the fluidic channels. Sample debris and aggregation issues may be further exacerbated by ex vivo drug treatment of primary cells given that sensitive cells may undergo necrosis or apoptosis leading to fragmentation (mechanism dependent).

Prior work demonstrates the capacity of MAR to define the therapeutic response of multiple myeloma patients to standard-of-care therapies (see, e.g., Cetin, A. E. et al. Determining therapeutic susceptibility in multiple myeloma by single-cell mass accumulation. Nat. Commun. 8, 1613 (2017)); however, solid tumors have remained difficult to measure. To determine whether active loading improves the feasibility of single-cell measurements on heterogeneous primary patient, sSMR devices with active loading were deployed to a preclinical laboratory setting. Using established protocols for isolating single cells from primary tissue samples (see, e.g., Filbin, M. G. et al. Developmental and oncogenic programs in H3K27M gliomas dissected by single-cell RNA-seq. Science 360, 331-335 (2018)) (FIG. 1A, Example 1), active loading enabled the sSMR to measure cell mass and MAR for a diverse range of clinical brain tissue and cancer samples exposed to either a standard-of-care therapy or experimental therapy currently in clinical trial (Table 5).

TABLE 5
Primary sample biomarkers and pathology

| Primary Tissue | | |
|---|---|---|
| Type | Drug Assessed | Notes |
| Normal Brain | Temozolomide | Normal brain was used as a negative control for drug |
| | | response as well as baseline mass accumulation, due to its |
| | | lack of in vitro cell replication. |
| Glioblastoma | Temozolomide | Temozolomide is part of the standard of care treatment for |
| | | glioblastoma. |
| | | Molecular analysis on this sample showed unmethylated |
| | | MGMT status, a biomarker associated with resistance to |
| | | temozolomide. |
| Recurrent | Abemaciclib | Abemaciclib is currently being tested in clinical trials of |
| Glioblastoma | | newly-diagnosed and recurrent glioblastoma. In tumor |
| | | cells, RB1 mutation/deletion is a known resistance |
| | | mechanism to abemaciclib. |
| | | Biomarker analyses did not show RB1 alteration in this |
| | | tumor sample. |
| Breast | Abemaciclib | Abemaciclib is a US Food and Drug Administration |
| adenocarcinoma | | (FDA)-approved therapy for the treatment of hormone |
| metastasis | | receptor (HR)-positive, human epidermal growth factor |
| | | receptor 2 (HER2)-negative advanced or metastatic breast |
| | | cancer. |
| | | Pathological analysis of this sample showed HR-positive |
| | | and HER2-negative statuses. |
| | RAD001 | RAD001 (everolimus) is another FDA-approved therapy |
| | | for the treatment of hormone receptor (HR)-positive, |
| | | human epidermal growth factor receptor 2 (HER2)- |
| | | negative advanced or metastatic breast cancer. |
| Non-small cell lung | Carboplatin | Carboplatin is part of the standard of care for the treatment |
| cancer (NSCLC) | | of metastatic NSCLC without activating EGFR, ROS1, |
| metastasis | | ALK or BRAF mutation. |
| | | Histomolecular analyses of this sample showed absence of |
| | | EGFR, ROS1, ALK or BRAF mutation. |
| Primary CNS | Ibrutinib | Ibrutinib is an FDA-approved therapy for the treatment of |
| Lymphoma | | several subtypes of lymphoma, and is currently evaluated |
| | | in primary CNS lymphoma within clinical trials. |

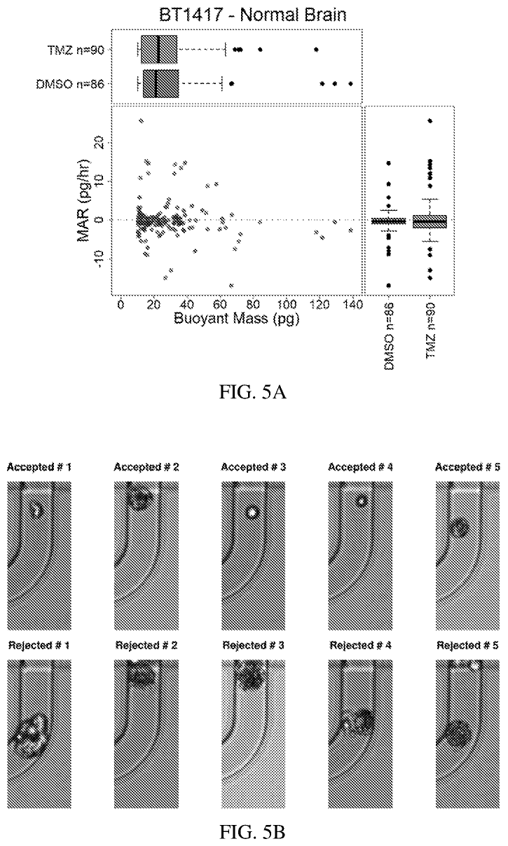

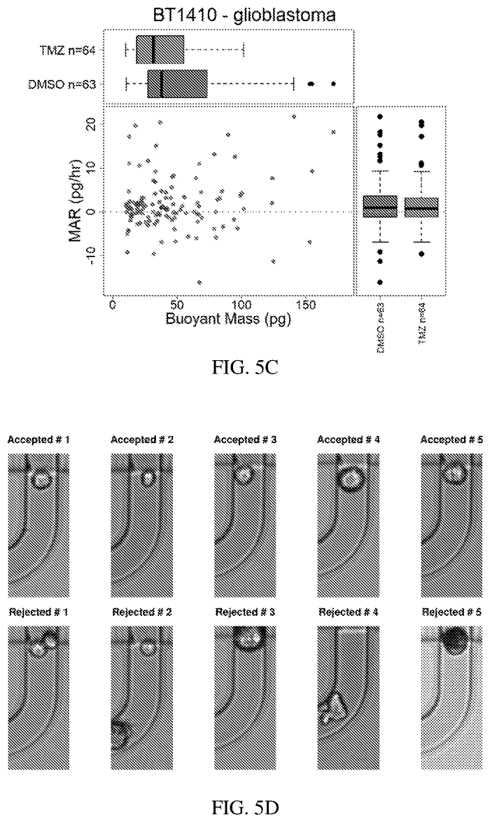

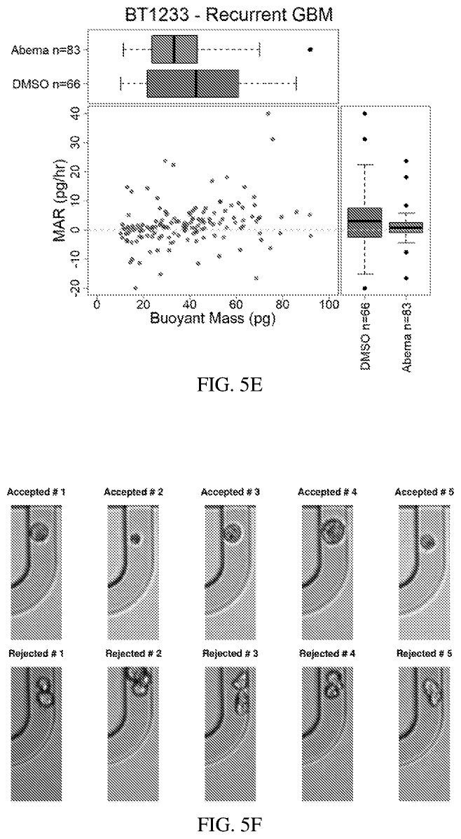

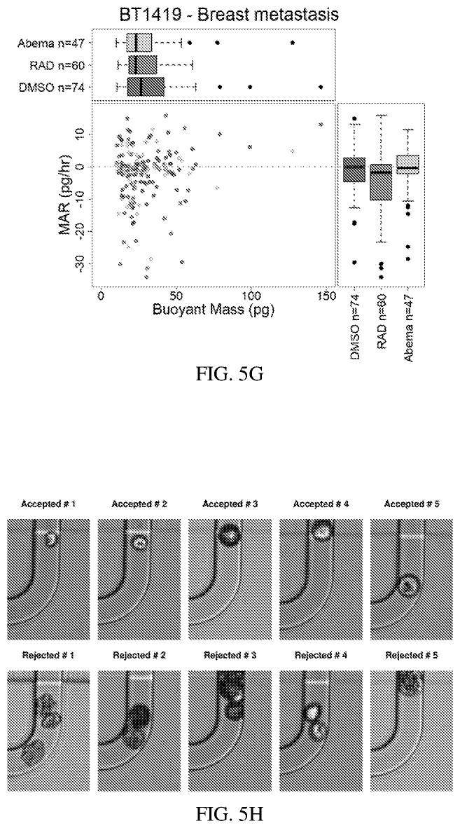

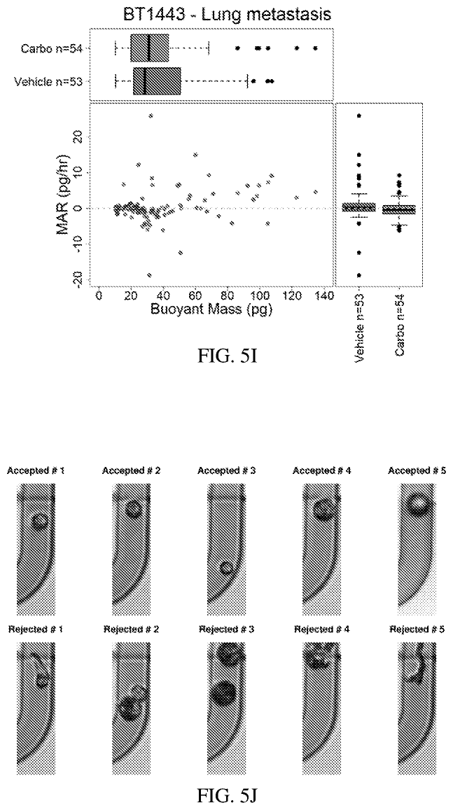

Measurements were obtained from five types of primary patient sample types, including: non-tumor brain tissue resected for a non-tumor condition (FIGS. 5A-5B, Table 6) (n=1); primary glioblastoma (FIGS. 5C-5D, Table 7) and recurrent glioblastoma (FIGS. 5E-5F, Table 8) (n=2); metastatic breast adenocarcinoma (FIGS. 5G-5H, Table 9) (n=1); metastatic non-small-cell lung cancer (FIGS. 5I-5J, Table 10) (n=1); and primary central nervous system (CNS) lymphoma (FIGS. 5K-5L, Table 11) (n=1). Measurements were made in a median time frame of 9 days following surgery (range of 2-18 days). Overall, mass and MAR were measured for 1092 cells with an average of 84 cells measured per condition over 13 conditions tested (FIGS. 1B-1C). The buoyant mass, MAR, and mass-normalized MAR of each drug-treated sample were compared with a paired vehicle control and significance was calculated using the Wilcoxon's signed-rank test.

TABLE 6
BT1417 - Normal brain information

| | Buoyant Mass | MAR | MAR per Mass |
|---|---|---|---|
| DMSO-TMZ p-value | 0.713 | 0.849 | 0.837 |

TABLE 7
BT1410 - Glioblastoma information

| | Buoyant Mass | MAR | MAR per Mass |
|---|---|---|---|
| DMSO-TMZ p-value | 0.0517 | 0.937 | 0.545 |

TABLE 8
BT1233 - Recurrent glioblastoma information

| | Buoyant Mass | MAR | MAR per Mass |
|---|---|---|---|
| DMSO-Abemaciclib | 0.164 | 0.0298 | 0.032 |
| p-value | | | |

TABLE 9
BT1419 - Breast metastasis information

| | Buoyant Mass | MAR | MAR per Mass |
|---|---|---|---|
| DMSO-RAD001 | 0.264 | 0.966 | 0.916 |
| p-value | | | |
| DMSO-Abemaciclib | 0.744 | 0.0240 | 0.0290 |
| p-value | | | |

TABLE 10
BT1443 - Lung metastasis information

| | Buoyant Mass | MAR | MAR per Mass |
|---|---|---|---|
| DMSO-Carboplatin | 0.998 | 0.0931 | 0.0251 |
| p-value | | | |

TABLE 11
BT1448 - CNS lymphoma information

| | Buoyant Mass | MAR | MAR per Mass |
|---|---|---|---|
| DMSO-Ibrutinib | 0.600 | 0.184 | 0.203 |
| p-value | | | |

The “rejection” capability of active loading was essential in performing sSMR measurements on the primary biopsies, as they contained a high amount of undesirable debris and cell aggregates that could prematurely terminate measurements by clogging the measurement channel. All six primary samples had images recorded and annotated of every particle accepted or rejected by the real-time Labview code. These images were manually reviewed and compared with the real-time determination to quantify the success rate at identifying unwanted particles in real time (e.g., as described in Example 1). For the six primary samples measured, the overall success rate for the real-time analysis code was 86% for correctly identifying single cells and allowing them to continue through the measurement channel.

No change in mass nor MAR was observed in cells isolated from the normal brain treated with TMZ (250 μM, 72 h). Normal brain tissue is non-proliferative, and was used as a negative control for both drug response and baseline in vitro growth. Similarly, no significant change was observed in the primary CNS lymphoma treated with ibrutinib (10 nM, 48 h), or the newly diagnosed glioblastoma treated with TMZ (250 μM, 8 days). Mass-normalized MAR was significantly reduced for the recurrent glioblastoma (p=0.032) treated with abemaciclib (1 μM, 72 h), breast metastasis (p=0.029) treated with abemaciclib (100 nM, 72 h), and the lung metastasis sample (p=0.025) treated with carboplatin (100 μM, 72 h). Active loading improved throughput and enabled measurement of previously incompatible tissues.

### Example 6—Single Cell Mass Accumulation Rate (MAR) in Response to Chemotherapy

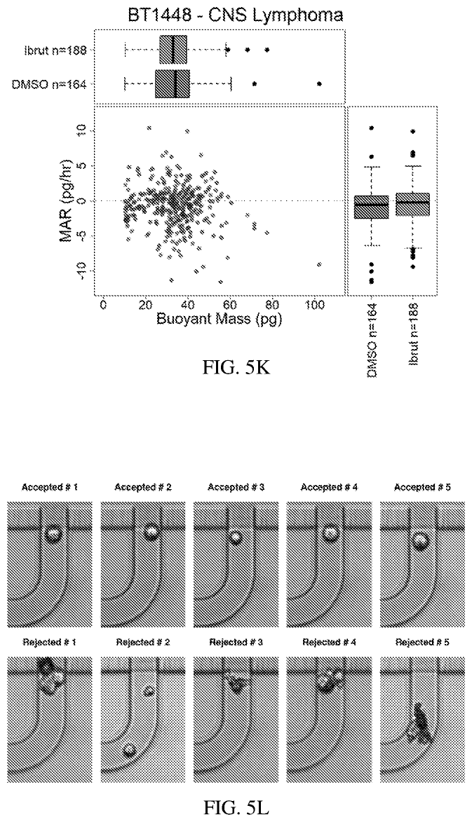

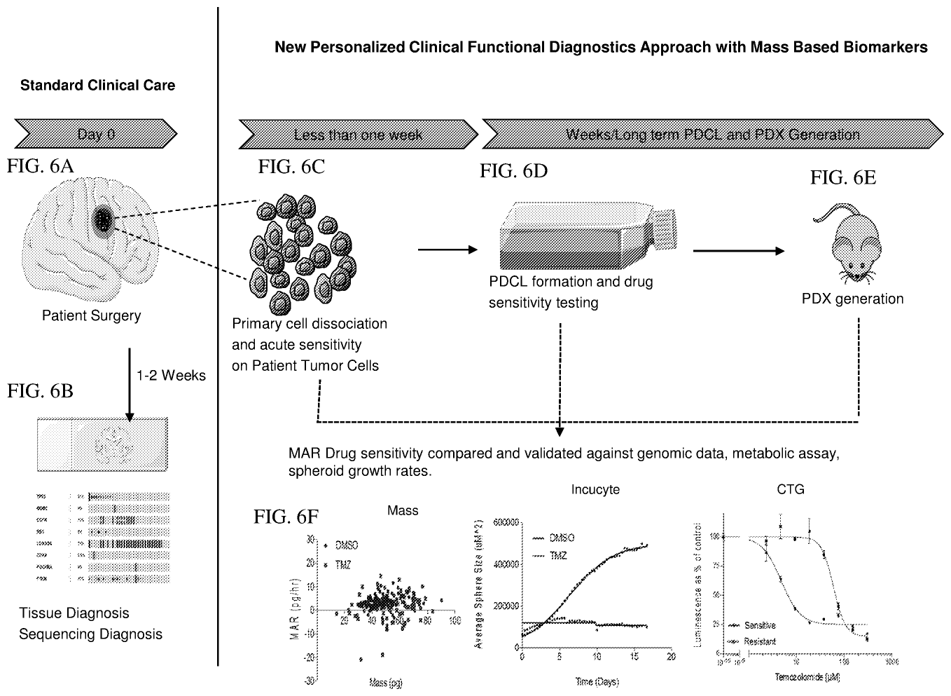

A single cell protocol for monitoring the mass accumulation rate (MAR) in response to chemotherapy (FIGS. 6A-6F). After tumor resection, the tissue is dissociated into single cells (FIG. 6A). The single cells from the dissociated tumor is measured within a week of resection using the mass/MAR of the single cells in response to chemotherapeutic agents (FIG. 6C). In addition to acute sensitivity testing, dissociated tumor cells from patient samples can also be grown long-term into robust cell lines (e.g., patient-derived cell lines, PDCLs), where high throughput experiments can be performed and analyzed relative to a more complete genomic background of the tissue (FIG. 6D). Because this assay is high throughput, the MAR can be measured with more conditions relative to acute patient samples. These PDCLs can be implanted in vivo to allow MAR measurements to be taken ex vivo from treated animals (e.g., mice). Metabolic readouts (e.g., CellTiter-Glo, CTG) and spheroid area analysis (e.g., IncuCyte) were conducted in parallel to the novel single cell MAR assays described herein (FIG. 6E).

The mass of cells treated with the chemotherapeutic agent Temozolomide (TMZ) showed a trend to decreased mass, and a smaller average spheroid size compared with cells treated with DMSO (FIG. 6E). Furthermore, the CellTiter-Glo assay was able to differentiate sensitive versus resistant single cells treated with TMZ (FIG. 6E). These data indicate that the single cell MAR assay can detect response to chemotherapy within a week after chemotherapeutic treatment, as well as predict which single cells will be sensitive and which cells will be resistant to treatment with a chemotherapeutic.

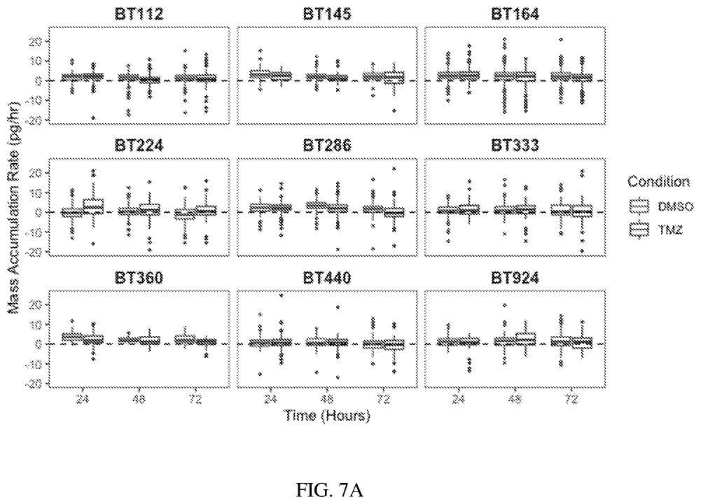

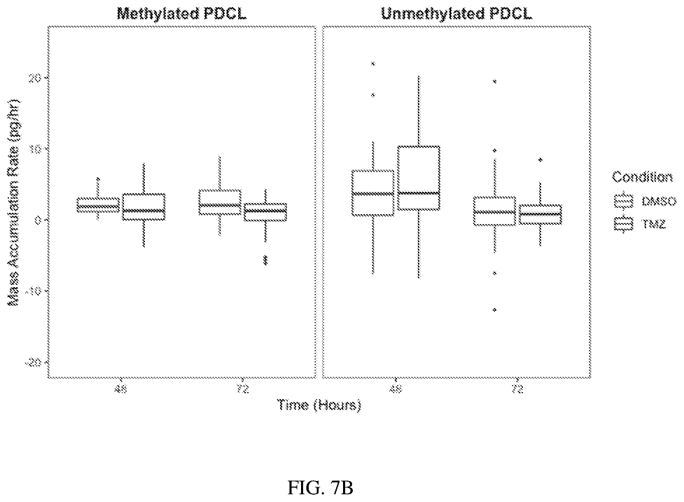

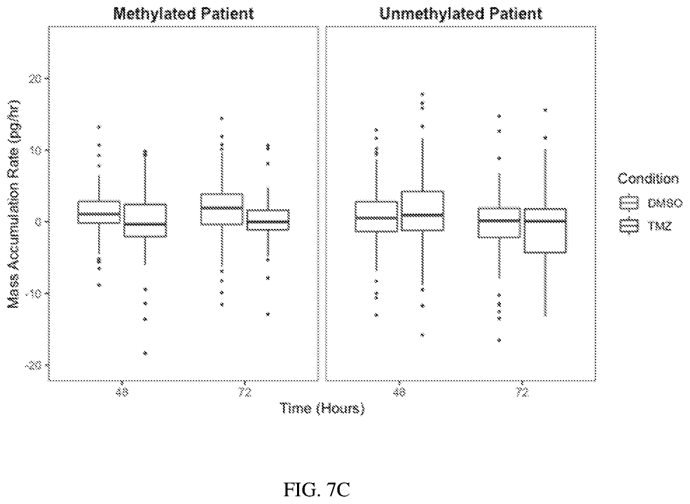

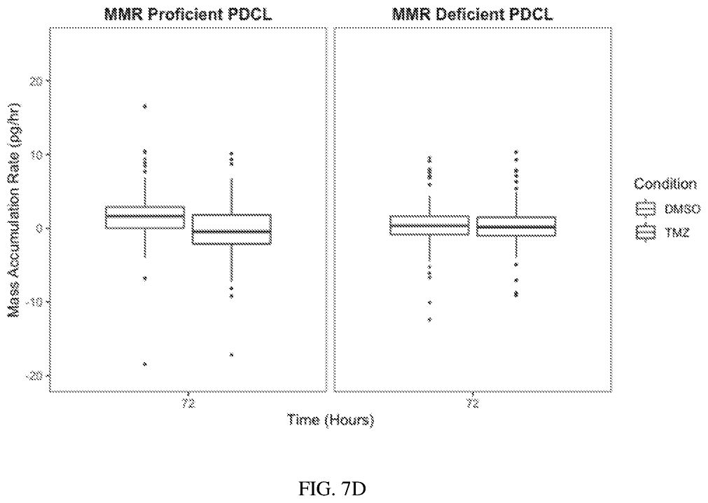

Single cell MAR experiments were conducted in both PDCLs and acute patient models (FIGS. 7A-7D). MAR experiments were conducted in cell lines with a wide range of cell morphology, genomic aberrations, and mutations (FIG. 7A). The single cell MAR assay detected changes within 24 hours after treatment with a chemotherapeutic (TMZ) compared with DMSO (FIG. 7A).

To determine if the single cell MAR assay is a predictive biomarker for cancer prognosis, single cells dissociated from patient-derived cell lines (PDCLs)/organoids and patient samples of glioblastoma multiforme (GBM) were treated with TMZ (FIGS. 7B, 7C). In response to TMZ, single cell measurements were able to detect a significant decrease of MAR in bulk populations in MGMT promoter methylated PDCLs compared to no significant decrease in unmethylated PDCLs. These results are consistent with clinical prognosis and median life expectancy of GBM patients, indicating that single cell MAR can serve as a predictive biomarker for cancer prognosis.

To determine if single cell MAR measurements are a predictive biomarker for resistance to chemotherapeutics, GBM PDCLs with varying proficiencies in mismatch repair (MMR) were treated with TMZ (FIG. 7D). There was a significant decrease in MAR in an MMR-proficient cell line, while the MAR of an MMR-deficient cell line was nearly identical to the control. These results indicate that single cell mass markers can be used as a detector of patients who may be resistant to chemotherapy due to MMR deficiency, which allows continued cellular proliferation in the presence of DNA damage.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

1. A method for evaluating sensitivity of a cancer cell to an anti-cancer reagent comprising:
(a) obtaining a tissue sample comprising primary cancer cells from a subject;
(b) dissociating the tissue sample into single primary cancer cells;
(c) contacting the single primary cancer cells with an anti-cancer reagent; and
(d) detecting the mass of the single primary cancer cell contacted with the anti-cancer reagent as it passes through a channel,
wherein the mass of the cell contacted with the anti-cancer reagent is compared to the normalized mass of a control cell that is not contacted with an anti-cancer reagent.

2. The method of claim 1, wherein if the mass of the cell contacted with the anti-cancer reagent is decreased compared to the control cell, the cancer cell is sensitive to the anti-cancer reagent.

3. The method of claim 1, wherein if the mass of the cell contacted with the anti-cancer reagent is the same or increased compared to the control cell, the cancer cell is resistant to the anti-cancer reagent.

4. The method of any one of claims 1-3, wherein steps (b)-(d) are performed within one hour to one month after step (a).

5. The method of any one of claims 1-4, wherein the single primary cancer cells of step (b) are cultured to produce patient-derived cell lines.

6. The method of claim 5, wherein the patient-derived cell lines are subjected to steps (c) and (d).

7. The method of claim 5 or claim 6, wherein the patient-derived cells lines are engrafted into a host subject, thereby generating a patient-derived xenograft.

8. The method of any one of claims 1-7, wherein dissociating the tissue sample comprises enzymatic and/or physical dissociation.

9. The method of any one of claims 1-8, wherein the anti-cancer reagent comprises radiation, small molecules, biologics, and/or DNA damaging agents.

10. The method of any one of claims 1-9, wherein the channel for detecting the mass of the single primary cancer cell is a measurement channel.

11. The method of claim 10, wherein the single cells are flowed into and through the measurement channel by active loading.

12. The method of claim 10 or claim 11, wherein the single cells are classified as single cells, cell aggregates, or debris in real-time before they are flowed into the measurement channel.

13. The method of claim 12, wherein the classification is at least 85% accurate at allowing only single cells into the measurement channel compared to manual classification.

14. The method of claim 12, wherein the classification is at least 50% accurate at rejecting cell aggregates and debris from the measurement channel compared to manual classification.

15. The method of any one of claims 1-14, wherein the contacting in step (c) is for 1-10 days.

16. A method for identifying an anti-cancer reagent comprising:
(a) obtaining a tissue sample comprising primary cancer cells from a subject;
(b) dissociating the tissue sample into single primary cancer cells;
(c) contacting the single primary cancer cells with a reagent; and
(d) detecting the mass of the single primary cancer cell contacted with the reagent as it passes through a channel,
wherein if the normalized mass of the cell contacted with the reagent is less than a control cell that is not contacted with the reagent, the reagent is an anti-cancer reagent.

17. The method of claim 16, wherein if the mass of the cell contacted with the anti-cancer reagent is the same or increased compared to the control cell, the reagent is not an anti-cancer reagent, with respect to that cell.

18. The method of claim 16 or claim 17, wherein steps (b)-(d) are performed within one hour to one month after step (a).

19. The method of any one of claims 16-18, wherein the single primary cancer cells of step (b) are cultured to produce patient-derived cells lines.

20. The method of claim 19, wherein the patient-derived cell lines are subjected to steps (c) and (d).

21. The method of claim 19 or claim 20, wherein the patient-derived cells lines are engrafted into a host subject, thereby generating a patient-derived xenograft.

22. The method of any one of claims 16-21, wherein dissociating the tissue sample comprises enzymatic and/or physical dissociation.

23. The method of any one of claims 16-22, wherein the reagent comprises small molecules, biologics, and/or DNA damaging agents.

24. The method of any one of claims 16-23, wherein the channel for detecting the mass of the single primary cancer cell is a measurement channel.

25. The method of claim 24, wherein the single cells are flowed into and through the measurement channel by active loading.

26. The method of claim 24 or claim 25, wherein the single cells are classified as single cells, cell aggregates, or debris in real-time before they are flowed into the measurement channel.

27. The method of claim 26, wherein the classification is at least 85% accurate at allowing single cells into the measurement channel compared to manual classification.

28. The method of claim 26, wherein the classification is at least 50% accurate at rejecting cell aggregates and debris from the measurement channel compared to manual classification.

29. The method of any one of claims 16-28, wherein the contacting in step (c) is for 1-10 days.

30. A method for evaluating sensitivity of a cancer cell to an anti-cancer reagent comprising:
(a) obtaining a tissue sample comprising primary cancer cells from a subject;
(b) dissociating the tissue sample into single primary cancer cells;
(c) culturing the single primary cancer cells to obtain patient-derived cell lines;
(d) contacting the patient-derived cell lines with an anti-cancer reagent;
(e) engrafting a host subject with the patient-derived cell lines contacted with the anti-cancer reagent;
(f) obtaining a tissue sample from the host subject;
(g) dissociating the tissue sample from the host subject into single cells; and
(h) detecting the mass of the single cells contacted with the anti-cancer reagent as they passes through a channel,
wherein the mass of the cell contacted with the anti-cancer reagent is compared to the normalized mass of a control cell that is not contacted with an anti-cancer reagent.

31. The method of claim 30, wherein if the mass of the cell contacted with the anti-cancer reagent is decreased compared to the control cell, the cancer cell is sensitive to the anti-cancer reagent.

32. The method of claim 30, wherein if the mass of the cell contacted with the anti-cancer reagent is the same or increased compared to the control cell, the cancer cell is resistant to the anti-cancer reagent.

33. The method of any one of claims 30-32, wherein steps (b)-(d) are performed within one hour to one month after step (a).

34. The method of any one of claims 30-33, wherein dissociating the tissue sample comprises enzymatic and/or physical dissociation.

35. The method of any one of claims 30-34, wherein the anti-cancer reagent comprises radiation, small molecules, biologics, and/or DNA damaging agents.

36. The method of any one of claims 30-35, wherein the channel for detecting the mass of the single primary cancer cell is a measurement channel.

37. The method of claim 36, wherein the single cells are flowed into and through the measurement channel by active loading.

38. The method of claim 36 or claim 37, wherein the single cells are classified as single cells, cell aggregates, or debris in real-time before they are flowed into the measurement channel.

39. The method of claim 38, wherein the classification is at least 85% accurate at allowing single cells into the measurement channel compared to manual classification.

40. The method of claim 38, wherein the classification is at least 50% accurate at rejecting cell aggregates and debris from the measurement channel compared to manual classification.

41. The method of any one of claims 30-41, wherein the contacting in step (c) is for 1-10 days.
